SEQUENCE LISTING (11 sequences)

SEQ ID NO: 1 - protein, 4 aa, Artificial Sequence
  Synthetic peptide
RGDS

SEQ ID NO: 2 - protein, 5 aa, Artificial Sequence
  Synthetic peptide
GRGDS

SEQ ID NO: 3 - protein, 6 aa, Artificial Sequence
  Synthetic peptide
GRGDSP

SEQ ID NO: 4 - protein, 7 aa, Artificial Sequence
  Synthetic peptide
GRGDSPK

SEQ ID NO: 5 - protein, 5 aa, Artificial Sequence
  Synthetic peptide
GRGDN

SEQ ID NO: 6 - protein, 6 aa, Artificial Sequence
  Synthetic peptide
GRGDNP

SEQ ID NO: 7 - protein, 8 aa, Artificial Sequence
  Synthetic peptide
GGGGRGDS

SEQ ID NO: 8 - protein, 5 aa, Artificial Sequence
  Synthetic peptide
GRGDK

SEQ ID NO: 9 - protein, 6 aa, Artificial Sequence
  Synthetic peptide
GRGDTP

SEQ ID NO: 10 - protein, 5 aa, Artificial Sequence
  Synthetic peptide
YRGDS

SEQ ID NO: 11 - protein, 12 aa, Artificial Sequence
  Synthetic peptide
HHLGGAKQAGDV

(19) United States
(12) Patent Application Publication
(10) Pub. No.: US 2016/0310615 A1
(43) Pub. Date: Oct. 27, 2016

(54) SPRAY ON HEMOSTATIC SYSTEM

(71) Applicant: CASE WESTERN RESERVE UNIVERSITY, Cleveland, OH (US)

(72) Inventor: Erin LAVIK, Cleveland Heights, OH (US)

(21) Appl. No.: 15/103,714

(22) PCT Filed: Dec. 11, 2014
(86) PCT No.: PCT/US2014/069821

§ 371 Date: Jun. 10, 2016

Related U.S. Application Data
(60) Provisional application No. 61/914,748, filed on Dec. 11, 2013.

(51) Int. Cl. A61K 47/48; A61K 47/34; A61K 47/22; A61K 45/06; A61K 38/08; A61K 38/12

(52) U.S. Cl. CPC A61K 47/48915; A61K 47/48907; A61K 38/08; A61K 38/12; A61K 47/22; A61K 45/06; A61K 47/34

(57) ABSTRACT

The invention provides for spray compositions comprising co-polymers comprising a core, water-soluble polymer and a peptide and a delivery solvent. The present invention provides for spray hemostatic systems that allow for quick and even distribution of hemostatic nanoparticles or synthetic platelets that reducing bleeding and improve outcomes in trauma. The invention provides for spray compositions comprising a co-block polymer coupled to a water soluble polymer, and a polymer delivery solvent. The invention provides for spray compositions which comprise nanoparticles that halve bleeding time in a femoral artery injury model, which allow for even distribution of the nanoparticles at a wound site and allow application to areas that are difficult to contact with other methods of administration.

This application claims priority benefit of U.S. Provisional Patent Application No. 61/914,748 filed Dec. 11, 2013, which is incorporated by reference herein in its entirety.

### STATEMENT OF GOVERNMENT INTEREST

This invention was made with government support under Grant Number CON114452 awarded by the National Institute of Health. The government has certain rights in the invention.

### FIELD OF INVENTION

The invention provides for spray compositions comprising a co-block polymer coupled a water soluble polymer, and a polymer delivery solvent.

### BACKGROUND

Hemorrhaging is also the first step in the injury cascade, for example, in the central nervous system (CNS). In both spinal cord and traumatic brain injuries, the first observable phenomena, regardless of mechanism of insult, is hemorrhaging. If one can stop the bleeding, presumably one can preserve tissue and improve outcomes. The primary mechanical insult is very often a small part of the injury. The secondary injury processes that occur over hours, days, and weeks following injury lead to progression and the poor functional outcomes. Stopping those secondary injury processes would mean preservation of greater amounts of tissue. Preservation of tissue means better functional outcomes.

Following injury, hemostasis is established through a series of coagulatory events. The critical steps in terms of platelets involve their activation, binding, and release of a host of growth factors and other molecules including fibrinogen. During vascular injury, collagen is exposed which triggers the activation of platelets. Platelet morphology shifts from a discoid to stellate, and they adhere to the exposed collagen. Once platelet aggregation begins, several inflammatory agents are released from their storage granules including adenosine diphosphate (ADP), which causes the surfaces of nearby circulating platelets to become adherent. Serotonin, epinephrine, and thromboxane A 2 further induce extreme vasoconstriction. The ultimate step, clot formation, is the conversion of fibrinogen, a large, soluble plasma protein produced by the liver and normally present in the plasma, into fibrin, an insoluble, threadlike molecule.

In severe injuries, these endogenous processes fall short and uncontrolled bleeding results. There have been a number approaches to augment these processes and induce hemostasis beyond the external methods. Platelet substitutes which either replace or augment the existing platelets have been pursued for a number of years (Blajchman, J. Thromb. Haemost. 1: 1637-41 (2003)). Administration of allogeneic platelets can help to halt bleeding; however, platelets have a short shelf life, and administration of allogeneic platelets can cause graft versus host disease, alloimmunization, and transfusion-associated lung injuries (Blajchman, J. Thromb. Haemost. 1: 1637-41 (2003)). Non-platelet alternatives including red blood cells modified with the Arg-Gly-Asp (RGD) sequence, fibrinogen-coated microcapsules based on albumin, and liposomal systems have been studied as coagulants (Siller-Matula et al., Thromb. Haemost. 100: 397-404 (2008)), but toxicity, thrombosis, and limited efficacy are major issues in the clinical application of these products (Frink et al., J. Biomed. Biotech. 2011: 979383 (2011)).

There are a number of approaches to augment hemostasis in the field and clinic including pressure dressings, absorbent materials such as QuikClot®, and intravenous (IV) infusion of activated recombinant factor VII (rFVIIa), but the former two are only applicable to exposed wounds, and rFVIIa has had both mixed results, requires refrigeration, and is expensive making it challenging to administer in the field or at the site of trauma. Clearly, a new approach to halt bleeding that is amenable to administration in the field is needed.

Spray on hemostatic systems have many advantages such as quick and even distribution over a broad coverage area. Spray on hemostatic systems can be easily applied to areas that are difficult to contact by swabs or bandages. There is a need for development of spray on systems hemostatic systems.

For a hemostat to be effective for complex trauma, the system needs to be non-toxic, stable when stored at room temperature (i.e. a medic's bag), have the potential for immediate. administration, and possess injury site-specific aggregation properties so as to avoid non-specific thrombosis. For this system to be clinically translatable, ideally it needs to be made with materials previously approved by the FDA. Practically, it also needs to be affordable.

### SUMMARY OF INVENTION

The present invention provides for spray hemostatic systems that allow for quick and even distribution of hemostatic nanoparticles or synthetic platelets that reducing bleeding and improve outcomes in trauma. The invention provides for spray compositions comprising a co-block polymer coupled to a water soluble polymer, and a polymer delivery solvent.

The invention provides for spray compositions which comprise nanoparticles that halve bleeding time in a femoral artery injury model, which allow for even distribution of the nanoparticles at a wound site and allow application to areas that are difficult to contact with other methods of administration. These nanoparticles act essentially as synthetic platelets and are stable at room temperature.

In one aspect, any of the spray compositions of the invention comprise a co-block polymer, wherein the co-block polymer is a nanoparticles comprising a core, a water soluble polymer and a peptide. In a particular embodiment, the water soluble polymer of the spray composition is attached to the core at a first terminus of the water soluble polymer. In addition, the peptide of the spray composition comprises an RGD amino acid sequence.

In another aspect, any of the spray compositions of the invention further comprise comprising a polycation. For example, the invention provides for spray compositions in which the polycation is positioned adjacent the co-block polymer and the water soluble polymer.

In any of the spray compositions of the invention, the co-block polymer is a diblock copolymer, a triblock copolymer, an amphiphilic block copolymer or a PEG block co-polymer. For examples, the co-block polymer is poly(lactide-co-glycolide acid (PLGA), polylactic acid (PLA), polyglycolide (PGA), polycaprolactone (PCL), poly (ε-caprolactone), poly-L-lysine (PLL) or combinations thereof.

This spray compositions of the invention are effective over a very wide polymer or nanoparticle concentration, e.g. at a concentration of 0.1% nanoparticles, 0.2% nanoparticles, 0.3% nanoparticles, 0.4% nanoparticles, 0.5% nanoparticles, 0.6% nanoparticles, 0.7% nanoparticles, 0.8% nanoparticles, 0.9% nanoparticles, 1.0% nanoparticles, 2.0% nanoparticles, 3.0% nanoparticles, 4.0% nanoparticles, 5.0% nanoparticles, 6.0% nanoparticles, 7.0% nanoparticles, 8.0% nanoparticles, 9.0% nanoparticles, 10% nanoparticles, 15% nanoparticles, 20% nanoparticles, 25% nanoparticles, 30% nanoparticles, 35% nanoparticles, 40% nanoparticles, 45% nanoparticles, 50% nanoparticles, 55% nanoparticles, 60% nanoparticles, 65% nanoparticles, 70% nanoparticles, 75% nanoparticles, 80% nanoparticles, 85% nanoparticles, 90% nanoparticles, 95% nanoparticles, or 99% nanoparticles.

This spray compositions of the invention may range from 0.1% to 99% nanoparticles, 0.1% to 0.25% nanoparticles, 0.1% to 0.5% nanoparticles, 0.1% to 0.75% nanoparticles, 0.1% to 10% nanoparticles, 0.5% to 0.75% nanoparticles, 0.5% to 1% nanoparticles, 0.5% to 25% nanoparticles, 1% to 10% nanoparticles, 1% to 20% nanoparticles, 1% to 30% nanoparticles, 5% to 10% nanoparticles, 5% to 25% nanoparticle, 5% to 50% nanoparticles, 10% to 20% nanoparticles, 10% to 30% nanoparticles, 10% to 50% nanoparticles, 10% to 75% nanoparticles, 20% to 30% nanoparticles, 20% to 40% nanoparticles, 20% to 50% nanoparticles, 20% to 60% nanoparticles, 20% to 30% nanoparticles, 20% to 40% nanoparticles, 20% to 50% nanoparticles, 20% to 75% nanoparticles, 20% to 80% nanoparticles, 30% to 40% nanoparticles, 30% to 40% nanoparticles, 30% to 50% nanoparticles, 30% to 60% nanoparticles, 30% to 70% nanoparticles, 30% to 80% nanoparticles, 30% to 90% nanoparticles, 40% to 50% nanoparticles, 40% to 60% nanoparticles, 40% to 70% nanoparticles, 40% to 80% nanoparticles, 40% to 90% nanoparticles, 50% to 60% nanoparticles, 50% to 75% nanoparticles, 50% to 80% nanoparticles, 50% to 90% nanoparticles, 50% to 95% nanoparticles, 60% to 70% nanoparticles, 60% to 75% nanoparticles, 60% to 80% nanoparticles, 60% to 85% nanoparticles, 60% to 90% nanoparticles, 60% to 95% nanoparticles, 70% to 75% nanoparticles, 70% to 80% nanoparticles, 70% to 85% nanoparticles, 70% to 90% nanoparticles, 70% to 95% nanoparticles, 75% to 90% nanoparticles, 75% to 95% nanoparticles, 75% to 98% nanoparticles, 80% to 90% nanoparticles, 80% to 85% nanoparticles, 80% to 90% nanoparticles, 80% to 95% nanoparticles, 80% to 98% nanoparticles, 80% to 99% nanoparticles or 90% to 98% nanoparticles.

In any of the spray compositions of the invention, the water soluble polymer is selected from the group consisting of polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1, 3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof.

For example, the invention provides for compositions comprising nanoparticles comprising the water soluble polymer PEG, such as PEG having an average molecular weight between 100 Da and 10,000 Da.

The invention provides for spray compositions wherein the polycation is selected from polylysine, polyarginine, polyornithine, polyhistidine, cationic polysaccharides, POLYBRENE® (1,5-dimethyl-1,5-diazaundecamethylene polymethobromide, hexadimethrine bromide), histone, myelin basic protein, polymyxin B sulfate, dodecyltrimethylammonium bromide, bradykinin, spermine, putrescine, cadaverine, octylarginine, cationic dendrimer, and synthetic peptides. In particular, the invention provides for spray compositions wherein the polycation is polylysine.

In any of the spray compositions of the invention, the nanoparticles comprise a peptide comprising a sequence selected from the group consisting of RGD, RGDS (SEQ ID NO: 1), GRGDS (SEQ ID NO: 2), GRGDSP (SEQ ID NO: 3), GRGDSPK (SEQ ID NO: 4), GRGDN (SEQ ID NO: 5), GRGDNP (SEQ ID NO: 6), GGGGRGDS (SEQ ID NO: 7), GRGDK (SEQ ID NO: 8), GRGDTP (SEQ ID NO: 9), cRGD, YRGDS (SEQ ID NO: 10) or variants thereof. The compositions of the invention may comprise a nanoparticle comprising a RGD peptide that is in a tandem repeat. The compositions of the invention may comprise nanoparticles comprising 2, 3, 4, 5, 6, 7, 8, 9, 10 or more copies of the RGD peptide or the nanoparticles comprising multiple copies of the RGD peptide. For example, the composition comprises nanoparticles comprising multiple copies of the RGD peptide and wherein all copies of the RGD peptide are the same or the composition comprises nanoparticles comprising multiple copies of the RGD peptide and wherein two copies of the RGD peptide have different sequences.

In any of the spray compositions of the invention, the polymer delivery solvent is a dipolar aprotic solvent. For example, the polymer delivery solvent is selected from the group consisting of dimethylsulfoxide (DMSO), N-Methyl-2-pyrrolidone (NMP), N,N dimethyl aceamide (DMF), and tetrahydrofuran (THF).

For example, the invention also provides for spray composition comprising a nanoparticle, the nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa), the composition optionally further comprising a poloxamer. The nanoparticles in the compositions of the invention are neutrally charged such as nanoparticles having a zeta potential of about −3.0 mV to about 3 mV.

The spray compositions of the invention include those in which the poloxamer is present at about 0.1% to about 60% of the composition. The invention also provides for compositions wherein the poloxamer is present at about 0.1% to about 40% of the composition.

In addition, spray compositions of the invention include those in which the poloxamer in the composition is present up to 50 times nanoparticle mass.

In any of the spray compositions of the invention, the poloxamer is a non ionic triblock copolymer comprising a structure -[hydrophilic polymer-hydrophobic polymer-hydrophilic polymer]n-.

In any of the spray composition of the invention, the poloxamer is -[polyethylene glycol-poly(propylene oxide)-polyethylene glycol]n-. For example, the poloxamer may be selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407 and Kolliphor P 188. In addition, the poloxamer may be selected from the group consisting of Pluronic® 10R5, Pluronic® 17R2, Pluronic® 17R, Pluronic® 25R2, Pluronic® 25R4, Pluronic® 31R1, Pluronic® F 108 Cast Solid Surfacta, Pluronic® F 108 NF, Pluronic® F 108 Pastille, Pluronic® F 108NF Prill Poloxamer 338, Pluronic® F 127, Pluronic® F 127 NF, Pluronic® F 127 NF 500 BHT Prill, Pluronic® F 127 NF Prill Poloxamer 407, Pluronic® F 38, Pluronic® F 38 Pastille, Pluronic® F 68, Pluronic® F 68 Pastille, Pluronic® F 68 LF Pastille, Pluronic® F 68 NF, Pluronic® F 68 NF Prill Poloxamer 188, Pluronic® F 77, Pluronic® F 77 Micropastille, Pluronic® F 87, Pluronic® F 87 NF, Pluronic® F 87 NF Prill Poloxamer 237, Pluronic® F 88, Pluronic® F 88 Pastille, Pluronic® F 98, Pluronic® L 10, Pluronic® L 101, Pluronic® L 121, Pluronic® L 31, Pluronic® L 35, Pluronic® L 43, Pluronic® L 44 NF, Poloxamer 124, Pluronic® L 61, Pluronic® L 62, Pluronic® L 62 LF, Pluronic® L 62D, Pluronic® L 64, Pluronic® L 81, Pluronic® L 92, Pluronic® L44 NF, Pluronic® N 3, Pluronic® P 103, Pluronic® P 104, Pluronic® P 105, Pluronic® P 123 Surfactant, Pluronic® P 65, Pluronic® P 84, and Pluronic® P 85.

In particular, the invention provides for a spray composition comprising a nanoparticle, the nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer having sufficient length to allow binding of the peptide to glycoprotein IIb/IIIa (GPIIb/IIIa), the composition further comprising a poly(acrylic acid), a polycation such as polylysine and a polymer delivery solvent. The nanoparticles of the spray composition may have a neutral charge or have a zeta potential of about −3.0 mV to about 3.0 mV.

In any of the spray compositions of the invention, the composition comprises nanoparticles having a spheroid shape and a diameter of less than 1 micron. For example, the nanoparticles has a diameter between 0.1 micron and 1 micron.

Alternatively, in any of the spray compositions of the invention, the composition comprises nanoparticles having a non-spheroid shape. For example, the nanoparticle is a rod, fiber or whisker. The nanoparticles may have an aspect ratio length to width of at least 3.

The invention provides for any of the foregoing spray compositions that are stable at room temperature for at least 14 days.

The invention also provides for any of the foregoing spray composition comprising nanoparticles having a core that is a crystalline polymer. In addition, any of the foregoing spray compositions comprise nanoparticles having a core that is a single polymer, a block copolymer, a triblock copolymer or a quadblock polymer. For example, the spray compositions of the invention comprise nanoparticles having a core comprising PLGA, PLA, PGA, (poly (ε-caprolactone) PCL, PLL or combinations thereof.

The invention provides for spray compositions comprising nanoparticles having a biodegradable core or alternatively a non-biodegradable core. In any of the compositions of the invention, the nanoparticles may have a solid core. For example, the invention provides for spray compositions comprising nanoparticles wherein the core is a material of gold, silver, platinum, aluminum, palladium, copper, cobalt, indium, nickel, ZnS, ZnO, Ti, TiO₂, Sn, SnO₂, Si, SiO₂, Fe, Fe⁺⁴, steel, cobalt-chrome alloys, Cd, CdSe, CdS, and CdS, titanium alloy, AgI, AgBr, HgI₂, PbS, PbSe, ZnTe, CdTe, In₂S₃, In₂Se₃, Cd₃P₂, Cd₃As₂, InAs, GaAs, cellulose or a dendrimer structure.

In any of the spray compositions of the invention, the composition comprises nanoparticles comprising a water soluble polymer attached to the core at a molar ratio of 0.1:1 to 1:10 or greater.

In any of the spray composition of the invention, the composition comprises nanoparticles further comprising a therapeutic compound. For example, the therapeutic compound is hydrophobic. Alternatively, the therapeutic compound is hydrophilic. The therapeutic compound may be covalently attached to the nanoparticle, non-covalently associated with the nanoparticle, associated with the nanoparticle through electrostatic interaction, or associated with the nanoparticle through hydrophobic interaction. The therapeutic compound may be a growth factor, a cytokine, a steroid, or a small molecule or an anti-cancer compound.

The invention provides for spray compositions which are pharmaceutical compositions, wherein the composition further comprises a pharmaceutically acceptable carrier, diluent or formulation.

The invention provides for methods of treating a condition in an individual comprising the step of administering any of the foregoing spray compositions to a patient in need thereof in an amount effective to treat the condition. For example, the invention provides for methods wherein the individual has a bleeding disorder and the spray composition is administered in an amount effective to reduce bleeding. In particular, the invention provide for methods of treating a bleeding disorder comprising the step of administering any of the foregoing spray compositions in an amount effective to reduce bleeding time by more than 15% compared to no administration or administration of saline. In these methods of the invention, the bleeding disorder may be a symptom of a clotting disorder, thrombocytopenia, wound healing disorder, trauma, blast trauma, a spinal cord injury or hemorrhaging.

The invention also provides for use of any of the spray compositions of the invention for the preparation of a medicament for the treatment of a condition wherein the medicament comprises the spray composition in an amount effective to treat the condition. For example, the invention provides for an use of any of the foregoing spray compositions of the invention for the preparation of a medicament for the treatment of a bleeding disorder wherein the medicament comprises the spray composition in an amount effective to reduce bleeding. The invention provides for an use of any of the foregoing compositions for the preparation of a medicament for the treatment of a bleeding disorder wherein the medicament comprise the spray composition in an amount effective to reduce bleeding time by more than 15% compared to no administration or administration of saline. In any of the uses of the invention, the medicament may be administered to treat a bleeding disorder that is a symptom of a clotting disorder, thrombocytopenia, a wound healing disorder, trauma, blast trauma, a spinal cord injury or hemorrhaging.

The invention also provides for spray compositions of the invention for treating a condition such as a bleeding disorder. The invention provides for spray compositions for treating a bleeding disorder wherein the bleeding disorder is a symptom of a clotting disorder, thrombocytopenia, a wound healing disorder, trauma, blast trauma, a spinal cord injury or hemorrhaging. The invention provides for spray compositions for the treatment of a bleeding disorder wherein the spray composition is administered in an mount effective to reduce bleeding time by more than 15% compared to no administration or administration of saline.

### BRIEF DESCRIPTION OF DRAWING

FIG. 1 provides a schematic of the PLGA-PLL nanoparticles of the invention.

FIG. 2A-FIG. 2B depicts the effect of nanoparticles on bleeding time in vitro

FIG. 3 depicts cumulative blood loss vs. lactated ringers control. The liver injury is made at time 0, and allowed to bleed freely. Blood is collected via suction. This curve represents cumulative blood loss averaged from 4 experiments. The majority of blood loss occurs in the first 5 minutes. The dotted lines denote SEM.

FIG. 4 depicts blood loss, divided into 4 time ranges, pre-administration (0-5 min, 380+/−59 ml), post-administration (5-15 min, 174+/−106 ml), post-infusion 1 (15-30 min, 150+/−111 ml), and post-infusion 2 (30-60 min, 70+/−95 ml). +/− represents S.D.

FIG. 5 depicts bleeding of pigs (n=5) over the first hours following liver lobe resection.

FIG. 6 depicts the surface of the liver following administration of spray on system showing the sealed surface.

FIG. 7 depicts removed section of pig liver showing the large vessels running through the liver.

FIG. 8 depicts rate of blood loss after administration of NP1 (0.1 mg/kg dose at 5 min post-injury). +/− represents S.D.

FIG. 9 depicts rate of blood loss after administration of NP100 (0.1 mg/kg dose at 5 min post-injury). +/− represents S.D.

FIG. 10 depicts percent of time animal spent at novel object. No statistical difference was detected. Active (5 animals), Control (5 animals) and LR (6 animals).

### DETAILED DESCRIPTION

Compositions comprising a functionalized nanoparticle is provided based on FDA-approved materials that has multiple uses. In various aspects, the nanoparticle reduces bleeding time at the site of injury, plays a role in hemostasis following trauma to the central nervous system (CNS) and provides a means for localized drug delivery.

Intravenous administration of hemostatic nanoparticles that target activated platelets have been investigated by a number of groups with some promise and a range of challenges. RGD conjugated red blood cells (RBCs) called thromboerythrocytes showed promise in vitro but did not significantly reduce prolonged bleeding times in thrombocytopenic primates. Fibrinogen-coated albumin microparticles, “Synthocytes” and liposomes used by others carrying the fibrinogen γ chain dodecapeptide (HHLGGAKQAGDV (SEQ ID NO: 11)) showed success in bleeding models in thrombocytopenic rabbits. However, Synthocytes were ineffective in treating bleeding in normal rabbits, and the liposomes do not appear to have yet been studied for this purpose.

The spray compositions of the invention are an improvement over intravenous administration of the nanoparticles of the invention because the spray allows for quick and even distribution of the nanoparticles at the site of the wound, which enhances wound healing and more efficiently mitigates bleeding. In addition, the spray compositions may be applied over a broad coverage area in a short period of time and allows for a controls and continuous supply to the affected area. Spray compositions allow for the synthetic platelets to be easily applied in awkward or hard to reach areas.

The experiments provided herein demonstrate that the hemostatic nanoparticles of the invention reduced bleeding in a number of models of trauma in rodents including femoral artery injuries, liver injuries, and blast traumas. In addition, these hemostatic nanoparticles following a blunt trauma liver injury in swine. The spray compositions evenly distribute the hemostatic nanoparticles of the invention which will allow of easy and quick application and enhance the ability to reduce bleeding.

The swine liver injury model has been developed to mimic non-compressible injuries sustained by military personnel and permits direct comparison to other hemostatic interventional studies. Briefly, the left lobe of the liver is isolated and hemisected followed by closure of the cavity and quantification of blood loss over time as a function of treatment regime coupled with continuous monitoring and blood analysis.

Initially, even low doses (0.2 mg/kg) of nanoparticles led to excessive bleeding. Testing of particles with uninjured swine demonstrated a strong complement-associated response which correlated with the charge on the nanoparticles. The nanoparticles of the invention were engineered to have a neutral charge, and this change resulted in a mitigation in the complement response induced by these particles.

The invention provides for spray compositions comprising a nanoparticle, polycation and a delivery solvent, the nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa). The compositions may further comprise a poloxamer.

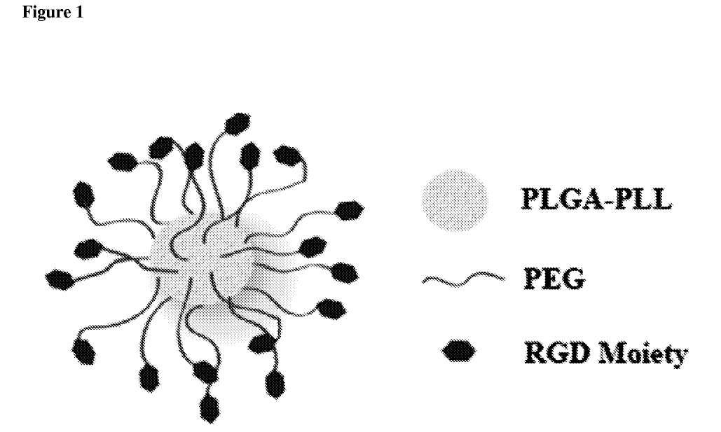

An exemplary nanoparticle of the invention is set out in FIG. 1 which comprises a PLGA-PLL nanosphere core (˜200 nm), PEG arms conjugated to the core at the first terminus and conjugated to RGD peptides conjugated to the PEG arms at the second terminus. This nanoparticle binds to activated platelets. The attributes of the nanoparticles of the invention include specificity for a vascular injury site, biocompatible and biodegradable. In addition, the nanoparticles may be stored dry at room temperature and have a rapid and easy administration.

### Nanoparticles

The disclosure provides a nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa). In various aspects, the peptide is linear or cyclic. It will be appreciated that in a composition comprising a plurality of nanoparticles of the disclosure, the composition is contemplated to include nanoparticles wherein all peptides are linear, all peptides are cyclic, or a mixture of linear and cyclic peptides is present.

Nanoparticles of the disclosure are temperature stable in that they maintain essentially the same structure and/or essentially the same function over a wide range of temperatures. By “essentially the same structure” and “essentially the same function,” the disclosure contemplates “essentially the same” to mean without a change that affects the ability of the nanoparticles to carry out its use at a dosage of plus or minus 10% of an original dosage, plus or minus 10% of an original dosage, plus or minus 10% of an original dosage, plus or minus 9% of an original dosage, plus or minus 8% of an original dosage, plus or minus 7% of an original dosage, plus or minus 6% of an original dosage, plus or minus 5% of an original dosage, or plus or minus 5%-10% of an original dosage. In various embodiments, the nanoparticles maintain essentially the same structure and/or essentially the same function at physiological temperature, regardless of the temperature at which the nanoparticles were produced. Nanoparticles that maintain essentially the same structure and/or essentially the same function at temperatures elevated well over physiological temperatures are also contemplated. The ability to maintain essentially the same structure and/or essentially the same function at elevated temperatures is important for any number of reasons, including, for example and without limitation, sterilization processes. On the other hand, nanoparticles which maintain essentially the same structure and/or essentially the same function at reduced temperatures are also contemplated. For example, nanoparticles that maintain essentially the same structure and/or essentially the same function at or below freezing temperatures are contemplated for formulations that require or benefit from long term storage. In various aspects the nanoparticle of the disclosure have a melting temperature over 35° C., over 40° C., over 45° C., over 50° C., over 55° C., over 60° C., over 65° C., over 70° C., over 71° C., over 72° C., over 73° C., over 74° C., over 75° C., over 76° C., over 77° C., over 78° C., over 79° C. or over 80° C.

The nanoparticle of all aspects of the disclosure are stable at room temperature for at least 5 days, at least 6 days, at least 7 days, at least 8 days, at least 9 days, at least 10 days, at least 11 days, at least 12 days, at least 13 days or at least 14 days or more.

Nanoparticle of the disclosure are contemplated to have any of a number of different shapes. The shape of the nanoparticle is in certain aspects, a function of the method of its production. In other aspects, the nanoparticle acquires a shaped that is formed before, during or after the process of its production. In various embodiments, nanoparticles are provided that have a spheroid shape. Spheroid nanoparticles (referred to herein as nanospheres) having various sizes are contemplated, wherein, for example nanoparticles having a diameter between 0.1 micron and 0.5 micron, between 0.2 micron and 0.4 micron, between 0.25 micron and 0.375 micron, between 0.3 micron and 0.375 micron, between 0.325 micron and 0.375 micron, between 0.12 microns and 0.22 microns, between 0.13 microns and 0.22 microns, between 0.14 microns and 0.22 microns, between 0.15 microns and 0.22 microns, between 0.16 microns and 0.22 microns, between 0.17 microns and 0.22 microns, between 0.18 microns and 0.22 microns, between 0.19 microns and 0.22 microns, between 0.20 microns and 0.22 microns, between 0.21 microns and 0.22 microns, between 0.12 microns and 0.21 microns, between 0.12 microns and 0.20 microns, between 0.12 microns and 0.19 microns, between 0.12 microns and 0.18 microns, between 0.12 microns and 0.17 microns, between 0.12 microns and 0.16 microns, between 0.12 microns and 0.15 microns, between 0.12 microns and 0.14 microns, or between 0.12 microns and 0.13 microns are contemplated. In various aspect, nanoparticles are contemplated having a diameter of 0.01 microns to 1.0 micron, 0.05 microns to 1.0 micron, 0.05 microns to 0.95 microns, 0.05 microns to 0.9 microns, 0.05 microns to 0.85 microns, 0.05 microns to 0.8 microns, 0.05 microns to 0.75 microns, 0.05 microns to 0.7 microns, 0.05 microns to 0.65 microns, 0.05 microns to 0.6 microns, 0.05 microns to 0.55 microns, 0.05 microns to 0.5 microns, 0.1 microns to 1 micron, 0.15 microns to 1.0 microns, 0.2 microns to 1 micron, 0.25 microns to 1.0 microns, 0.3 microns to 1 micron, 0.35 microns to 1.0 microns, 0.4 microns to 1 micron, 0.45 microns to 1.0 microns, or 0.5 microns to 1 micron. In compositions of nanoparticles provided by the disclosure, the spherical nanoparticles are homogenous in that that all have the same diameter, or they are heterogeneous in that at least two nanoparticles in the composition have different diameters.

Nanoparticle are also provided which are non-spheroid. Other nanoparticles include those having a rod, fiber or whisker shape. In rod, fiber or whisker embodiments, the nanoparticle has a sufficiently high aspect ratio to avoid, slow or reduce the rate of clearance from circulation.

Aspect ratio is a term understood in the art, a high aspect ratio indicates a long and narrow shape and a low aspect ratio indicates a short and thick shape.

Nanoparticle of the disclosure are contemplated with an aspect ratio length to width of at least 3, of at least 3.5, of at least 4.0, of at least 4.5, of at least 5.0, of at least 5.5, of at least 6.0, of at least 6.5, of at least 7.0, of at least 7.5, of at least 8.0, of at least 8.5, of at least 9.0, of at least 9.5, of at least 10.0 or more. In a composition of nanoparticles contemplated, the nanoparticles have, in one embodiment, identical aspect ratios, and in alternative embodiments, at least two nanoparticles in the composition have different aspects ratios. Composition of nanoparticles are also characterized by having, on average, essentially the same aspect ratio. “Essentially the same” as used in this instance indicated that variation in aspect ratio of about 10%, about 9%, about 8%, about 7% about 6% or up to about 5% is embraced. In still other aspects, a composition of nanoparticles is provided wherein the nanoparticles in the composition have an aspect ratio of between about 1% and 200%, between about 1% and 150%, between about 1% and 100%, between about 1% and about 50%, between about 50% and 200%, between about 100% and 200%, and between about 150% and 200%. Alternatively, the nanoparticles in the composition have an aspect ratio from about X % to Y %, wherein X from 1 up to 100 and Y is from 100 up to 200.

The disclosure also provides a plurality of nanoparticles. In compositions comprising a plurality of spherical nanoparticles provided by the disclosure, nanoparticles in the plurality have an average diameter between 0.1 micron and 0.5 micron, between 0.2 micron and 0.4 micron, between 0.25 micron and 0.375 micron, between 0.3 micron and 0.375 micron, between 0.325 micron and 0.375 micron, about 0.12 micron, about 0.13 micron, about 0.14 micron, about 0.15 micron, about 0.16 micron, about 0.17 micron, about 0.18 micron, about 0.19 micron, about 0.20 micron, about 0.21 micron, about 0.22 micron, about 0.23 micron, about 0.24 micron, about 0.25 micron, about 0.26 micron, about 0.27 micron, about 0.28 micron, about 0.29 micron, about 0.30 micron, about 0.31 micron, about 0.32 micron, about 0.33 micron, about 0.34 micron, about 0.35 micron, about 0.36 micron, about 0.37 micron, about 0.38 micron, about 0.39 micron, about 0.40 micron, about 0.41 micron, about 0.42 micron, about 0.43 micron, about 0.44 micron, about 0.45 micron, about 0.46 micron, about 0.47 micron, about 0.48 micron, about 0.49 micron, about 0.50 micron, about 0.41 micron, about 0.52 micron, about 0.53 micron, about 0.54 micron, about 0.55 micron, about 0.56 micron, about 0.57 micron, about 0.58 micron, about 0.59 micron, about 0.60 micron, about 0.61 micron, about 0.62 micron, about 0.63 micron, about 0.64 micron, about 0.65 micron, about 0.66 micron, about 0.67 micron, about 0.68 micron, about 0.69 micron, about 0.70 micron, about 0.71 micron, about 0.72 micron, about 0.73 micron, about 0.74 micron, about 0.75 micron, about 0.76 micron, about 0.77 micron, about 0.78 micron, about 0.79 micron, about 0.80 micron, about 0.81 micron, about 0.82 micron, about 0.83 micron, about 0.84 micron, about 0.85 micron, about 0.86 micron, about 0.87 micron, about 0.88 micron, about 0.89 micron, about 0.90 micron, about 0.91 micron, about 0.92 micron, about 0.93 micron, about 0.94 micron, about 0.95 micron, about 0.96 micron, about 0.97 micron, about 0.98 micron, about 0.99 micron, about 1.0 micron, or more.

In various aspects, the plurality of spherical nanoparticles are characterized in that greater than 75%, greater than 80%, greater than 85%, greater than 90%, greater than 95%, greater than 96%, greater than 97%, greater than 98%, or greater than 99% of all nanoparticles have a diameter between 0.1 micron and 0.5 micron, between 0.2 micron and 0.4 micron, between 0.25 micron and 0.375 micron, between 0.3 micron and 0.375 micron, between 0.325 micron and 0.375 micron, between 0.12 microns and 0.22 microns, between 0.13 microns and 0.22 microns, between 0.14 microns and 0.22 microns, between 0.15 microns and 0.22 microns, between 0.16 microns and 0.22 microns, between 0.17 microns and 0.22 microns, between 0.18 microns and 0.22 microns, between 0.19 microns and 0.22 microns, between 0.20 microns and 0.22 microns, between 0.21 microns and 0.22 microns, between 0.12 microns and 0.21 microns, between 0.12 microns and 0.20 microns, between 0.12 microns and 0.19 microns, between 0.12 microns and 0.18 microns, between 0.12 microns and 0.17 microns, between 0.12 microns and 0.16 microns, between 0.12 microns and 0.15 microns, between 0.12 microns and 0.14 microns, between 0.12 microns and 0.13 microns, 0.01 microns to 1.0 micron, 0.05 microns to 1.0 micron, 0.05 microns to 0.95 microns, 0.05 microns to 0.9 microns, 0.05 microns to 0.85 microns, 0.05 microns to 0.8 microns, 0.05 microns to 0.75 microns, 0.05 microns to 0.7 microns, 0.05 microns to 0.65 microns, 0.05 microns to 0.6 microns, 0.05 microns to 0.55 microns, 0.05 microns to 0.5 microns, 0.1 microns to 1 micron, 0.15 microns to 1.0 microns, 0.2 microns to 1 micron, 0.25 microns to 1.0 microns, 0.3 microns to 1 micron, 0.35 microns to 1.0 microns, 0.4 microns to 1 micron, 0.45 microns to 1.0 microns, or 0.5 microns to 1 micron.

The nanoparticles in the compositions of the invention are neutrally charged such a nanoparticles having a zeta potential of about −3.0 mV to about 3.0 mV. For example, the nanoparticles have a zeta potential ranging from −3.0 mV to about 2.9 mV, about −3.0 mV to about 2.7 mV, −3.0 mV to about 2.5 mV, about −3.0 mV to about 2.3 mV, about −3.0 mV to about 2.0 mV, about −3.0 mV to about 1.7 mV, about −3.0 mV to about 1.5 mV, −3.0 mV to about 1.3 mV, about −3.0 mV to about 1.0 mV, about −3.0 mV to about 0.75 mV, about −3.0 mV to about 0.5 mV, about −3.0 mV to about 0.25 mV, about −3.0 mV to about 0.1 mV, about −3.0 mV to about 0.05 mV, about −3.0 mV to about 0.125 mV, about −3.0 mV to about 0 mV, about −3.0 mV to about −0.125, about −3.0 mV to about −0.25 mV, about −3.0 to about −0.50 mV, about −3.0 mV to about −0.75, about −3.0 mV to about −1.0 mV, about −3.0 mV to about −1.3 mV, about −3.0 mV to about −1.5 mV, about −3.0 mV to about −1.7 mV, about −3.0 mV to about −2.0 mV, about −3.0 mV to about −2.3 mV, −3.0 mV to about −2.7 mV, −3.0 mV to about 3 mV, −2.5 to about 3.0 mV, −2.5 mV to about 2.9 mV, about −2.5 mV to about 2.7 mV, −2.5 mV to about 2.5 mV, about −2.5 mV to about −2.5 mV, about −2.5 mV to about 2.0 mV, about −2.5 mV to about 1.7 mV, about −2.5 mV to about 1.5 mV, −2.5 mV to about 1.3 mV, about −2.5 mV to about 1.0 mV, about −2.5 mV to about 0.75 mV, about −2.5 mV to about 0.5 mV, about −2.5 mV to about 0.25 mV, about −2.5 mV to about 0.1 mV, about −2.5 mV to about 0.05 mV, about −2.5 mV to about 0.125 mV, about −2.5 mV to about 0 mV, about −2.5 mV to about −0.125, about −2.5 mV to about −0.25 mV, about −2.5 to about −0.50 mV, about −2.5 mV to about −0.75, about −2.5 mV to about −1.0 mV, about −2.5 mV to about −1.3 mV, about −2.5 mV to about −1.5 mV, about −2.5 mV to about −1.7 mV, about −2.5 mV to about −2.0 mV, about −2.5 mV to about −2.3 mV, −2.0 to about 3.0 mV, −2.0 mV to about 2.9 mV, about −2.0 mV to about 2.7 mV, −2.0 mV to about 2.0 mV, about −2.5 mV to about 2.5 mV, about −2.0 mV to about 2.0 mV, about −2.0 mV to about 1.7 mV, about −2.0 mV to about 1.5 mV, −2.0 mV to about 1.3 mV, about −2.0 mV to about 1.0 mV, about −2.0 mV to about 0.75 mV, about −2.0 mV to about 0.5 mV, about −2.0 mV to about 0.25 mV, about −2.0 mV to about 0.1 mV, about −2.0 mV to about 0.05 mV, about −2.0 mV to about 0.125 mV, about −2.0 mV to about 0 mV, about −2.0 mV to about −0.125, about −2.0 mV to about −0.25 mV, about −2.0 to about −0.50 mV, about −2.0 mV to about −0.75, about −2.0 mV to about −1.0 mV, about −2.0 mV to about −1.3 mV, about −2.0 mV to about −1.5 mV, about −2.0 mV to about −1.7 mV, about −1.5 to about 3.0 mV, −1.5 mV to about 2.9 mV, about −1.5 mV to about 2.7 mV, −1.5 mV to about 2.5 mV, about −1.5 mV to about 2.5 mV, about −1.5 mV to about 2.0 mV, about −1.5 mV to about 1.7 mV, about −1.5 mV to about 1.5 mV, −1.5 mV to about 1.3 mV, about −1.5 mV to about 1.0 mV, about −1.5 mV to about 0.75 mV, about −1.5 mV to about 0.5 mV, about −1.5 mV to about 0.25 mV, about −1.5 mV to about 0.1 mV, about −1.5 mV to about 0.05 mV, about −2.5 mV to about 0.125 mV, about −1.5 mV to about 0 mV, about −1.5 mV to about −0.125, about −1.5 mV to about −0.25 mV, about −1.5 to about −0.50 mV, about −1.5 mV to about −0.75, about −0.5 mV to about −1.0 mV, about −1.5 mV to about −1.3 mV, −1.0 to about 3.0 mV, −1.0 mV to about 2.9 mV, about −1.0 mV to about 2.7 mV, −1.0 mV to about 2.5 mV, about −1.0 mV to about 2.5 mV, about −1.0 mV to about 2.0 mV, about −1.0 mV to about 1.7 mV, about −1.0 mV to about 1.5 mV, −1.0 mV to about 1.3 mV, about −1.0 mV to about 1.0 mV, about −1.0 mV to about 0.75 mV, about −1.0 mV to about 0.5 mV, about −1.0 mV to about 0.25 mV, about −1.0 mV to about 0.1 mV, about −1.0 mV to about 0.05 mV, about −1.0 mV to about 0.125 mV, about −1.0 mV to about 0 mV, about −1.0 mV to about −0.125, about −1.0 mV to about −0.25 mV, about −1.0 to about −0.50 mV, about −1.0 mV to about −0.75, about −1.0 mV to about −1.0 mV, −0.5 mV to about 3.0 mV, −0.5 mV to about 2.9 mV, about −0.5 mV to about 2.7 mV, −0.5 mV to about 2.5 mV, about −0.5 mV to about 2.5 mV, about −0.5 mV to about 2.0 mV, about −0.5 mV to about 1.7 mV, about −0.5 mV to about 1.5 mV, −0.5 mV to about 1.3 mV, about −0.5 mV to about 1.0 mV, about −0.5 mV to about 0.75 mV, about −0.5 mV to about 0.5 mV, about −0.5 mV to about 0.25 mV, about −0.5 mV to about 0.1 mV, about −0.5 mV to about 0.05 mV, about −0.5 mV to about 0.125 mV, about −0.5 mV to about 0 mV, about −0.5 mV to about −0.125, about −0.5 mV to about −0.25 mV, 0 mV to about 3.0 mV, 0 mV to about 2.9 mV, about 0 mV to about 2.7 mV, 0 mV to about 2.5 mV, about 0 mV to about 2.5 mV, about 0 mV to about 2.0 mV, about 0 mV to about 1.7 mV, about 0 mV to about 1.5 mV, 0 mV to about 1.3 mV, about 0 mV to about 1.0 mV, about 0 mV to about 0.75 mV, about 0 mV to about 0.5 mV, about 0 mV to about 0.25 mV, about 0 mV to about 0.1 mV, about 0 mV to about 0.05 mV, about 0 mV to about 0.125 mV, 0.25 mV to about 3.0 mV, 0.25 mV to about 2.9 mV, about 0.25 mV to about 2.7 mV, 0.25 mV to about 2.5 mV, about 0.25 mV to about 2.5 mV, about 0.25 mV to about 2.0 mV, about 0.25 mV to about 1.7 mV, about 0.25 mV to about 1.5 mV, 0.25 mV to about 1.3 mV, about 0.25 mV to about 1.0 mV, about 0.25 mV to about 0.75 mV, about 0.25 mV to about 0.5 mV, 0.5 mV to about 3.0 mV, 0.5 mV to about 2.9 mV, about 0.5 mV to about 2.7 mV, 0.5 mV to about 2.5 mV, about 0.5 mV to about 2.5 mV, about 0.5 mV to about 2.0 mV, about 0.5 mV to about 1.7 mV, about 0.5 mV to about 1.5 mV, 0.5 mV to about 1.3 mV, about 0.5 mV to about 1.0 mV, about 0.5 mV to about 0.75 mV, 0.75 mV to about 3.0 mV, 0.75 mV to about 2.9 mV, about 0.75 mV to about 2.7 mV, 0.75 mV to about 2.5 mV, about 0.75 mV to about 2.5 mV, about 0.75 mV to about 2.0 mV, about 0.75 mV to about 1.7 mV, about 0.75 mV to about 1.5 mV, 0.75 mV to about 1.3 mV, about 0.75 mV to about 1.0 mV, 1.0 mV to about 3.0 mV, 1.0 mV to about 2.9 mV, about 1.0 mV to about 2.7 mV, 1.0 mV to about 2.5 mV, about 1.0 mV to about 2.5 mV, about 1.0 mV to about 2.0 mV, about 1.0 mV to about 1.7 mV, about 1.0 mV to about 1.5 mV, 1.0 mV to about 1.3 mV, 1.5 mV to about 3.0 mV, 1.5 mV to about 2.9 mV, about 1.5 mV to about 2.7 mV, 1.5 mV to about 2.5 mV, about 1.5 mV to about 2.5 mV, about 1.5 mV to about 2.0 mV, about 1.5 mV to about 1.7 mV, 1.7 mV to about 3.0 mV, 1.7 mV to about 2.9 mV, about 1.7 mV to about 2.7 mV, 1.7 mV to about 2.5 mV, about 1.7 mV to about 2.5 mV, about 1.7 mV to about 2.0 mV, 2.0 mV to about 3.0 mV, 2.0 mV to about 2.9 mV, about 2.0 mV to about 2.7 mV, 2.0 mV to about 2.5 mV, about 2.0 mV to about 2.5 mV, 2.5 mV to about 3.0 mV, 2.5 mV to about 2.9 mV, about 2.5 mV to about 2.7 mV, 2.7 mV to about 3.0 mV or 2.7 mV to about 2.9 mV.

The disclosure further provides nanoparticles of essentially any shape are formed using microfabrication processes well known and routinely practiced in the art. In microfabrication methods, size and shape of the nanoparticles are predetermined by design.

### Core

A nanoparticle as described above is provided wherein the core is a polymer. In various aspects, the core is a crystalline polymer. “Crystalline” as used herein and understood in the art is defined to mean an arrangement of molecules in regular three dimensional arrays. In other aspects, the polymers are semi-crystalline which contain both crystalline and amorphous regions instead of all molecule arranged in regular three dimensional arrays. In various aspects, the core is a single polymer, a block copolymer, or a triblock copolymer. In specific aspects, the core comprises PLGA, PLA, PGA, (poly (ε-caprolactone) PCL, PLL, cellulose, poly(ethylene-co-vinyl acetate), polystyrene, polypropylene, dendrimer-based polymers or combinations thereof.

In various aspects, the core is biodegradable or non-biodegradable, or in a plurality of nanoparticles, combinations of biodegradable and non-biodegradable cores are formulated in contemplated. In various aspects, the core is solid, porous or hollow. In pluralities of nanoparticles, it is envisioned that mixtures of solid, porous and/or hollow cores are included.

Nanoparticle of any aspect of the disclosure include those wherein the core alternatively is a material selected from the group consisting of gold, silver, platinum, aluminum, palladium, copper, cobalt, indium, nickel, ZnS, ZnO, Ti, TiO₂, Sn, SnO₂, Si, SiO₂, Fe, Fe⁺⁴, steel, cobalt-chrome alloys, Cd, CdSe, CdS, and CdS, titanium alloy, AgI, AgBr, HgI₂, PbS, PbSe, ZnTe, CdTe, In₂S₃, In₂Se₃, Cd₃P₂, Cd₃As₂, InAs, GaAs, cellulose or a dendrimer structure.

Hydrogel core are also provided. In one aspect, the hydrogel core provides a higher degree of temperature stable, be less likely to shear vessels and induce non-specific thrombosis and allow formation of larger nanoparticles.

### Water Soluble Polymers

A nanoparticle of the disclosure is provided wherein the water soluble polymer is selected from the group consisting of polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1, 3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof. In a plurality of nanoparticles contemplated by the disclosure, each nanoparticle is contemplated, in various aspects, to have the same water soluble polymer, or alternatively, at least two nanoparticles in the plurality each have a different water soluble polymer attached thereto.

In a specific aspect, the nanoparticle of the disclosure is one wherein the water soluble polymer is PEG. For nanoparticles in this aspect, the PEG has an average molecular weight between 100 Da and 10,000 Da, 500 Da and 10,000 Da, 1000 Da and 10,000 Da, 1500 Da and 10,000 Da, 2000 Da and 10,000 Da, 2500 Da and 10,000 Da, 3000 Da and 10,000 Da, 3500 Da and 10,000 Da, 4000 Da and 10,000 Da, 4500 Da and 10,000 Da, 5000 Da and 10,000 Da, 5500 Da and 10,000 Da, 1000 Da and 9500 Da, 1000 Da and 9000 Da, 1000 Da and 8500 Da, 1000 Da and 8000 Da, 1000 Da and 7500 Da, 1000 Da and 7000 Da, 1000 Da and 6500 Da, or 1000 Da and 6000 Da. Alternatively, the nanoparticle is one in which PEG has an average molecular weight of about 100, Da, 200 Da, 300 Da, 400 Da, 1000 Da, 1500 Da, 3000 Da, 3350 Da, 4000 Da, 4600 Da, 5,000 Da, 8,000 Da, or 10,000 Da. In a plurality of nanoparticles, it is contemplated that each nanoparticle is attached to a PEG water soluble polymer of the same molecular weight, or in the alternative, at least two nanoparticles in the plurality are each attached to a PEG water soluble polymer which do not have the same molecular weight.

The nanoparticle of the disclosure includes those wherein the water soluble polymer is attached to the core at a molar ratio of 0.1:1, 0.2:1, 0.3:1, 0.4:1, 0.5:1, 0.6:1, 0.7:1, 0.8:1, 0.9:1, 1:1, 1:2, 1:3, 1:4, 1:5, 1:6, 1:7, 1:8, 1:9, 1:10 or greater. In various aspect, a plurality is proved wherein the water soluble polymer to core ratio is identical for each nanoparticle in the plurality, and in alternative aspect, at least two nanoparticles in the plurality have different water soluble polymer to core ratios.

The degree to which a nanoparticle is associated with a water soluble polymer is, in various aspects, determined by the route of administration chosen.

### Peptides

The nanoparticle of the disclosure is characterized by having a peptide associated therewith. In various aspects of the disclosure. The peptide is linear or cyclic. In specific embodiments, the peptide comprises a core sequence selected from the group consisting of RGD, RGDS (SEQ ID NO: 1), GRGDS (SEQ ID NO: 2), GRGDSP (SEQ ID NO: 3), GRGDSPK (SEQ ID NO: 4), GRGDN (SEQ ID NO: 5), GRGDNP (SEQ ID NO: 6), GGGGRGDS (SEQ ID NO: 7), GRGDK (SEQ ID NO: 8), GRGDTP (SEQ ID NO: 9), cRGD, YRGDS (SEQ ID NO: 10) or variants thereof. Variants are used herein include peptides have a core sequence as defined herein and one or more additional amino acid residues attached at one or both ends of the core sequence, a peptide having a core sequence as defined herein but wherein one or more amino acid residues in the core sequence is substituted with an alternative amino acid residue; the alternative amino acid residue being a naturally-occurring amino acid residue or a non-naturally-occurring amino acid residue, a peptide having a core sequence as defined herein but wherein one or more amino acid residues in the core sequence is deleted, or combinations thereof, wherein the additional amino acid residue, the amino acid substitution, the amino acid deletion or the combination of changes does (or do) not essentially alter the activity of the nanoparticle. “Essentially” as used in this aspect is the same as the meaning described elsewhere in the disclosure.

In various aspects, the RGD peptide is in a tandem repeat arrangement and in embodiments of this aspects, 1, 2, 3, 4, 5, 6, 7, 8, 9, 10 or more copies of the RGD peptide are contemplated. In another aspect, multiple copies of an RGD peptide are attached to the same nanoparticle, albeit not in a random repeat arrangement.

In various aspects wherein the nanoparticle is associated with multiple RGD peptides, the disclosure provide a nanoparticle wherein all copies of the RGD peptide are the same, as wells as aspects wherein two of the RGD peptide have different sequences.

In a plurality of nanoparticles contemplated, embodiments are provided wherein the RGD peptide (or multiple copies of RGD peptides) are identical on each nanoparticle in the plurality. In alternative aspects, at least two nanoparticles in the plurality each are associated with one or more distinct RGD peptides.

In various aspect, the number of peptides on a nanoparticle, i.e., the peptide density, affects platelet aggregation.

### Poloxamers

The spray compositions of the invention may comprise a poloxamer which is a stabilizer. The poloxamer reduces or eliminates aggregation of the neutrally-charged nanoparticles. Poloxamers are non-ionic triblock copolymers with a hydrophobic block at the center (poly(propylene oxide)) and two PEG groups at the ends. Poloxamers are also known as Pluronics in the field. Any poloxamer or pluroinic may be used in the compositions of the invention.

For example, the invention provides for spray compositions wherein the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407 and Kolliphor P 188, Pluronic® 10R5, Pluronic® 17R2, Pluronic® 17R, Pluronic® 25R2, Pluronic® 25R4, Pluronic® 31R1, Pluronic® F 108 Cast Solid Surfacta, Pluronic® F 108 NF, Pluronic® F 108 Pastille, Pluronic® F 108NF Prill Poloxamer 338, Pluronic® F 127, Pluronic® F 127 NF, Pluronic® F 127 NF 500 BHT Prill, Pluronic® F 127 NF Prill Poloxamer 407, Pluronic® F 38, Pluronic® F 38 Pastille, Pluronic® F 68, Pluronic® F 68 Pastille, Pluronic® F 68 LF Pastille, Pluronic® F 68 NF, Pluronic® F 68 NF Prill Poloxamer 188, Pluronic® F 77, Pluronic® F 77 Micropastille, Pluronic® F 87, Pluronic® F 87 NF, Pluronic® F 87 NF Prill Poloxamer 237, Pluronic® F 88, Pluronic® F 88 Pastille, Pluronic® F 98, Pluronic® L 10, Pluronic® L 101, Pluronic® L 121, Pluronic® L 31, Pluronic® L 35, Pluronic® L 43, Pluronic® L 44 NF, Poloxamer 124, Pluronic® L 61, Pluronic® L 62, Pluronic® L 62 LF, Pluronic® L 62D, Pluronic® L 64, Pluronic® L 81, Pluronic® L 92, Pluronic® L44 NF, Pluronic® N 3, Pluronic® P 103, Pluronic® P 104, Pluronic® P 105, Pluronic® P 123 Surfactant, Pluronic® P 65, Pluronic® P 84, and Pluronic® P 85.

In addition, other triblock copolymers that have PEG on the ends and a more hydrophobic middle group may be used as a stabilizer in the compositions as long as the polymer is soluble in water. Exemplary triblock copolymers include polymers having the ABA structure where A is PEG or PVA or another water soluble polymer and B is PLA, PGA, PLGA, polypropylene, poly(propylene oxide), a polyamide, polystyrene, polybutadiene, are examples. Alternatively, the triblock copolymer having the ABA structure where B is PEG or any of the water soluble polymers and A is any of the hydrophobic or water insoluble polymers.

The spray compositions of the invention may comprise about 0.1% poloxamer, about 0.2% poloxamer, about 0.3% poloxamer, about 0.4% poloxamer, about 0.5% poloxamer, about 0.6% poloxamer, about 0.7% poloxamer, about 0.8% poloxamer, about 0.9% poloxamer, about 1% poloxamer, about 2% poloxamer, about 3% poloxamer, about 4% poloxamer, about 5% poloxamer, about 6% poloxamer, about 7% poloxamer, about 8% poloxamer, about 9% poloxamer, about 10% poloxamer, about 11% poloxamer, about 12% poloxamer, about 13% poloxamer, about 14% poloxamer, about 15% poloxamer, about 16% poloxamer, about 17% poloxamer, about 18% poloxamer, about 19% poloxamer, about 20% poloxamer, about 21% poloxamer, about 22% poloxamer, about 23% poloxamer, about 24% poloxamer, about 25% poloxamer, about 26% poloxamer, about 27% poloxamer, about 28% poloxamer, about 29% poloxamer, about 30% poloxamer, about 31% poloxamer, about 32% poloxamer, about 33% poloxamer, about 34% poloxamer, about 35% poloxamer, about 36% poloxamer, about 37% poloxamer, about 38% poloxamer, about 39% poloxamer, about 40% poloxamer, about 41% poloxamer, about 42% poloxamer, about 43% poloxamer, about 44% poloxamer, about 45% poloxamer, about 46% poloxamer, about 47% poloxamer, about 48% poloxamer, about 49% poloxamer, about 40% poloxamer, about 51% poloxamer, about 52% poloxamer, about 53% poloxamer, about 54% poloxamer, about 55% poloxamer, about 56% poloxamer, about 57% poloxamer, about 58% poloxamer, about 59% poloxamer or about 60% poloxamer.

The invention provides for spray composition wherein the poloxamer is present at about 0.1% to about 60% of the composition, or at about 0.1% to about 55% of the composition, or at about 0.1% to about 50% of the composition, or at about 0.1% to about 45% of the composition, or at about 0.1% to about 40% of the composition, or at about 0.1% to about 35% of the composition, or at about 0.1% to about 30% of the composition, or at about 0.1% to about 25% of the composition, or at about 0.1% to about 20% of the composition, or at about 0.1% to about 15% of the composition, or at about 0.1% to about 12% of the composition, or at about 0.1% to about 10% of the composition, or at about 0.1% to about 5% of the composition, or at about 0.1% to about 1% of the composition, or at about 0.1% to about 0.5% of the composition, about 0.5% to about 60% of the composition, or at about 0.5% to about 55% of the composition, or at about 0.5% to about 50% of the composition, or at about 0.5% to about 45% of the composition, or at about 0.1% to about 40% of the composition, or at about 0.5% to about 35% of the composition, or at about 0.5% to about 30% of the composition, or at about 0.5% to about 25% of the composition, or at about 0.5% to about 20% of the composition, or at about 0.5% to about 15% of the composition, or at about 0.5% to about 12% of the composition, or at about 0.5% to about 10% of the composition, or at about 0.5% to about 5% of the composition, or at about 0.5% to about 1% of the composition, or about 1% to about 60% of the composition, or at about 1% to about 55% of the composition, or at about 1% to about 50% of the composition, or at about 1% to about 45% of the composition, or at about 1% to about 40% of the composition, or at about 1% to about 35% of the composition, or at about 1% to about 30% of the composition, or at about 1% to about 25% of the composition, or at about 1% to about 20% of the composition, or at about 1% to about 15% of the composition, or at about 1% to about 12% of the composition, or at about 1% to about 10% of the composition, or at about 1% to about 5% of the composition, or about 5% to about 60% of the composition, or at about 5% to about 55% of the composition, or at about 5% to about 50% of the composition, or at about 5% to about 45% of the composition, or at about 5% to about 40% of the composition, or at about 5% to about 35% of the composition, or at about 5% to about 30% of the composition, or at about 5% to about 25% of the composition, or at about 5% to about 20% of the composition, or at about 5% to about 15% of the composition, or at about 5% to about 12% of the composition, or at about 5% to about 10% of the composition, or about 10% to about 60%, or at about 10% to about 50% of the composition, or at about 10% to about 45% of the composition, or at about 10% to about 40% of the composition, or at about 10% to about 35% of the composition, or at about 10% to about 30% of the composition, or at about 10% to about 25% of the composition, or at about 10% to about 20% of the composition, or at about 10% to about 15% of the composition, or at about 10% to about 12% of the composition, or about 20% to about 60% of the composition, or at about 20% to about 50% of the composition, or at about 20% to about 45% of the composition, or at about 20% to about 40% of the composition, or at about 20% to about 35% of the composition, or at about 20% to about 30% of the composition, or at about 20% to about 25% of the composition, or about 30% to about 60%, or at about 30% to about 50% of the composition, or at about 30% to about 45% of the composition, or at about 30% to about 40% of the composition, or at about 30% to about 35% of the composition, or about 40% to about 60%, or at about 40% to about 50% of the composition, or at about 40% to about 45% of the composition, or about 45% to about 60%, or at about 45% to about 50% of the composition, or at about 50% to about 60% of the composition.

The invention provides for spray composition wherein the poloxamer is present up to 50 times nanoparticle mass, or up to 40 times nanoparticle mass, or up to 35 time nanoparticle mass, or up to 30 times nanoparticle mass, or up to 25 times nanoparticle mass, or up to 20 times nanoparticle mass, or up to 15 times nanoparticle mass, or up to 10 times nanoparticle mass, or up to 9 times nanoparticle mass, or up to 8 times nanoparticle mass, or up to 7 times nanoparticle mass, or up to 6 times nanoparticle mass, or up to 5 times nanoparticle mass.

Other stabilizers which do not impart a negative charge on the spray composition may be used in the compositions of the invention, such as poly(acrylic acid), poloxamer such as poloxamer 188 or PEG.

Other Compounds with the Nanoparticle

A nanoparticle of the disclosure is also contemplated further comprising a therapeutic compound. In various aspects, the therapeutic compound is hydrophobic and in still other aspects, the therapeutic compound is hydrophilic. A nanoparticle of the disclosure is provided wherein the therapeutic compound is covalently attached to the nanoparticle, non-covalently associated with the nanoparticle, associated with the nanoparticle through electrostatic interaction, or associated with the nanoparticle through hydrophobic interaction. In various embodiments, the therapeutic compound is a growth factor, a cytokine, a steroid, or a small molecule. Embodiments are contemplated wherein more than one therapeutic compound is associated with a nanoparticle. In this aspect, each therapeutic compounds associated with the nanoparticle is the same, or each therapeutic compound associated with the nanoparticle is different. In a plurality of nanoparticles provided by the disclosure, each nanoparticle in the plurality is associated with the same therapeutic compound or compounds, or in the alternative, at least two nanoparticles in the plurality is each associated with one or more different therapeutic compounds.

In various aspects, the therapeutic compound is a anti-cancer compound, and in specific embodiments, the therapeutic compound is selected from the group consisting of: an alkylating agents including without limitation nitrogen mustards, such as mechlor-ethamine, cyclophosphamide, ifosfamide, melphalan and chlorambucil; nitrosoureas, such as without limitation carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as without limitation busulfan; triazines such as dacarbazine (DTIC); antimetabolites including folic acid analogs such as methotrexate and trimetrexate; pyrimidine analogs such as without limitation 5-fluorouracil, fluorodeoxyuridine, gemcitabine, cytosine arabinoside (AraC, cytarabine), 5-azacytidine, 2,2′-difluorodeoxycytidine; purine analogs such as without limitation 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including without limitation antimitotic drugs such as paclitaxel; vinca alkaloids including without limitation vinblastine (VLB), vincristine, and vinorelbine, taxotere, estramustine, and estramustine phosphate; epipodophylotoxins such as without limitation etoposide and teniposide; antibiotics such as without limitation actimomycin D, daunomycin (rubidomycin), doxorubicin, mitoxantrone, idarubicin, bleomycins, plicamycin (mithramycin), mitomycinC, and actinomycin; enzymes such as without limitation L-asparaginase; biological response modifiers such as without limitation interferon-alpha, IL-2, G-CSF and GM-CSF; miscellaneous agents including without limitation platinum coordination complexes such as cisplatin and carboplatin; anthracenediones such as without limitation mitoxantrone; substituted urea such as without limitation hydroxyurea; methylhydrazine derivatives including without limitation N-methylhydrazine (MIH) and procarbazine; adrenocortical suppressants such as without limitation mitotane (o,p′-DDD) and aminoglutethimide; hormones and antagonists including without limitation adrenocorticosteroid antagonists such as prednisone and equivalents, dexamethasone and aminoglutethimide; progestin such as without limitation hydroxyprogesterone caproate, medroxyprogesterone acetate and megestrol acetate; estrogen such as without limitation diethylstilbestrol and ethinyl estradiol equivalents; antiestrogen such as without limitation tamoxifen; androgens including testosterone propionate and fluoxymesterone/equivalents; antiandrogens such as without limitation flutamide, gonadotropin-releasing hormone analogs and leuprolide; non-steroidal antiandrogens such as without limitation flutamide; folate inhibitors; tyrosine kinase inhibitors such as without limitation AG1478, and radiosensitizing compounds.

In various aspects, the therapeutic compound is selected from the group consisting of AG1478, acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, droloxifene, dromostanolone, duazomycin, edatrexate, eflomithine, elsamitrucin, enloplatin, enpromate, epipropidine, epirubicin, erbulozole, esorubicin, estramustine, estramustine, etanidazole, etoposide, etoposide, etoprine, fadrozole, fazarabine, fenretinide, floxuridine, fludarabine, fluorouracil, flurocitabine, fosquidone, fostriecin, fulvestrant, gemcitabine, gemcitabine, hydroxyurea, idarubicin, ifosfamide, ilmofosine, interleukin II (IL-2, including recombinant interleukin II or rIL2), interferon alpha-2a, interferon alpha-2b, interferon alpha-n1, interferon alpha-n3, interferon beta-1a, interferon gamma-I b, iproplatin, irinotecan, lanreotide, letrozole, leuprolide, liarozole, lometrexol, lomustine, losoxantrone, masoprocol, maytansine, mechlorethamine hydrochloride, megestrol, melengestrol acetate, melphalan, menogaril, mercaptopurine, methotrexate, methotrexate, metoprine, meturedepa, mitindomide, mitocarcin, mitocromin, mitogillin, mitomalcin, mitomycin, nitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, puromycin, pyrazofurin, riboprine, rogletimide, safingol, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, torernifene, trestolone, triciribine, triethylenemelamine, trimetrexate, triptorelin, tubulozole, uracil mustard, uredepa, vapreotide, verteporlin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin. These and other antineoplastic therapeutic agents are described, for example, in Goodman & Gilman's The Pharmacological Basis of Therapeutics, McGraw-Hill Professional, 10th ed., 2001.

In various aspects, the therapeutic compound is an anti-inflammatory selected from the group consisting of glucocorticoids; kallikrein inhibitors; corticosteroids (e.g. without limitation, prednisone, methylprednisolone, dexamethasone, or triamcinalone acetinide); anti-inflammatory agents (such as without limitation noncorticosteroid anti-inflammatory compounds (e.g., without limitation ibuprofen or flubiproben)); vitamins and minerals (e.g., without limitation zinc); anti-oxidants (e.g., without limitation carotenoids (such as without limitation a xanthophyll carotenoid like zeaxanthin or lutein)) and agents that inhibit tumor necrosis factor (TNF) activity, such as without limitation adalimumab (HUMIRA®), infliximab REMICADE®), certolizumab (CIMZIA®), golimumab (SIMPONI®), and etanercept (ENBREL®).

In various aspects, the therapeutic compound is M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor ÿ, cytokine-induced eutrophils chemotactic factor 1, cytokine-induced eutrophils, chemotactic factor 2, cytokine-induced neutrophils chemotactic factor 2, endothelial cell growth factor, endothelin 1, epithelial-derived eutrophils attractant, glial cell line-derived neutrophic factor receptor 1, glial cell line-derived neutrophic factor receptor 2, growth related protein, growth related protein, growth related protein y, growth related protein, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor, nerve growth factor nerve growth factor receptor, neurotrophin-3, neurotrophin-4, pre-B cell growth stimulating factor, stem cell factor, stem cell factor receptor, transforming growth factor, transforming growth factor, transforming growth factor, transforming growth factor 2, transforming growth factor ÿ, transforming growth factor, transforming growth factor β, latent transforming growth factor β, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, intracellular sigma peptide (ISP), and chimeric proteins and biologically or immunologically active fragments thereof.

Methods are also provided for with anticoagulation drugs. Including, for example and without limitation, plavix, aspirin, warfarin, heparin, ticlopidine, enoxaparin, Coumadin, dicumarol, acenocoumarol, citric acid, lepirudin and combinations thereof.

Methods in this aspect overcome the effects of these anticoagulant drugs which would be extremely helpful in surgery.

### Spray Compositions

The spray composition of the invention comprise a polymer delivery solvent that is a dipolar aprotic solvent such as dimethylsulfoxide (DMSO), N-Methyl-2-pyrrolidone (NMP), N,N dimethyl aceamide (DMF) or tetrahydrofuran (THF). The compositions of the present invention are preferably applied in a metered dose over a predetermined surface area.

The spray compositions of the invention may be administered using a spray system, an air brush system or a syringe type system. Alternatively, the compositions may be administered to the subject using an endoscope or other laproscopic device. Finally, the compositions of the invention may be administered via catheter. For example, the air brush system has broad applications including: administering the synthetic platelets to junctional injuries such as groin injuries in which the bleeding cannot be controls with typical pressure dressings, GI bleeds, and bleeding following trauma such as gross blunt trauma associated bleeds (e.g liver lacerations, other major organ lacerations.)

The spray dispenser of the invention includes any device that releases an aerosol, mist or film at the site of injury to efficiently reduce bleeding. Any device designed to produce a fine spray of liquid that can be suspended in a gas such as the atmosphere may be used to administer the spray composition. The dispenser commonly consists of a container that holds the composition under pressure to be applied as a liquefied gas propellant. When a valve is released, the propellant forces the composition through an atomizer and out of the dispenser in the form of a fine spray. For example, the spray composition of the invention may be administered by an atomizer, pump, sprayer or dropper.

Optionally, the spray compositions of the invention are formulated to be dispensed as an aerosol. For example, the composition may comprise a propellant in an amount to provide from about 10% to about 90% (w/w) of the composition. The propellant can be any pharmaceutically acceptable propellant which provides a suitable pressure within an aerosol dispenser, preferably a pressure of from about 20 p.s.i.g. to about 130 p.s.i.g. Preferred propellants include hydrocarbons, for example, propane, butane, isobutane, or dimethylether; hydrofluorocarbons and hydrochlorofluorocarbons, for example, dichlorodifluoromethane (P12), trichloromonofluoromethane (P11), dichlorofluoroethane, monochlorodifluoromethane (P22), dichlorotetrafluoroethane (P114), difluoroethane (P152a), tetrafluoroethane (134a), heptafluoropropane (P227b); or compressed gases, for example, nitrogen or carbon dioxide.

The aerosol dispenser is preferably a conventional aerosol having a conventional atomizer or metered spray aerosol valve. For example, the pump dispenser is preferably a conventional can or bottle having a conventional metered spray pump. Preferably, the aerosol dispenser has an all position valve having a covering that permits spraying when the dispenser is held at any angle. In this way, horizontal bottom surfaces, as well as horizontal top surfaces and vertical surfaces, can be sprayed. The valve actuator can be any actuator which produces a spray at the nozzle.

A preferred valve actuator is a mechanical breakup actuator, which employs mechanical forces rather than expansion and evaporation of the propellant to produce a spray. A typical mechanical breakup actuator has a conical or cylindrical swirl chamber with an inlet channel oriented perpendicular to the axis thereof. This structure imparts a swirling motion to the aerosol mixture upon discharge. The swirling motion occurs around the axis of the swirl chamber forming a thin conical film of discharged mixture, which breaks into droplets as it leaves the swirl chamber and travels in the direction of the axis thereof. The result is a fine, soft, dispersed spray which can be easily controlled to produce a stable thin film of even thickness completely contacting the application site. In dispensing a spray composition of the invention, the dispenser is typically held about 1 to 5 inches (2.5 to 12.5 cm) from the application site and produces a film of even thickness. The dispensers used in the present invention are preferably compact units, which can be conveniently used for quick and easy application of the composition over a large surface area.

The spray compositions of the invention have a drying time that allows for the reduction in bleeding time at the site of injury. It is important that the drying time allow for sufficient time to spread the formulation into a thin layer on the skin surface before the formulation is solidified, leading to poor skin contact. If the formulation dries too slowly, the subject may have to wait a long time before resuming normal activities (e.g. putting clothing on, working, etc.) that may remove un-solidified formulation. Thus, it is desirable that the drying time of the formulation under standard skin and ambient conditions be longer than about 15 seconds but shorter than about 15 minutes, such as from about 0.5 minutes to about 5 minutes, from about 15 seconds minutes to about 2 minutes, from about 1 minutes to about 3 minutes, from about 0.5 minutes to about 2.5 minutes, from about 2 minutes to about 10 minutes.

The spray compositions of the invention can be stored in a pressurized container and be sprayed on the skin surface with the help of the propellant. Some hydrofluorocarbons commonly used as propellants in pharmaceutical or domestic industries can work in this design. More specifically, the propellants may include, but not limited to dimethyl ether, butane, 1,1, difluoroethane, 1,1,1,2 tetrafluorethane, 1,1,1,2,3,3,3-heptafluoropropane, 1,1,1,3,3,3 hexafluoropropane, or a mixture thereof. The formulation may also be expelled out of the container and applied on the skin via a manual pump. Formulations comprising a these room temperature gaseous volatile solvents are expected to dry much faster. Spraying the formulation onto the skin suffering from neuropathic pain can avoid touching the skin with an applicator which can cause severe pain in the sometimes hypersensitive skin.

The spray compositions of the invention may also comprise a solubilizer. Exemplary solubilizers include a copolymer of dimethylamine ethyl methacrylate and a neutral methacrylic acid ester (Eudragit E100®, USP/NF); surfactants, for example, sodium lauryl sulphate; polyhydric alcohols, for example, propylene glycol or polyethylene glycol; vitamin E, vitamin E TPGS (tocopheryl polyethylene glycol 1000 succinate) and labrasol; or any two or more of the above in combination. Preferably, the solubilizer is a copolymer of dimethylamine ethyl methacrylate and a neutral methacrylic acid ester (Eudragit E100®) in combination with, a non-ionic copolymer of methyl methacrylate and butyl methacrylate (Plastoid B®). The solubilizers serve to dissolve the drug in the chosen vehicle. Many of the solubilizers also enhance percutaneous penetration of drug and/or act as humectants.

### Pharmaceutical Compositions

The invention provides for pharmaceutical spray compositions comprising a polymer or nanoparticle of the invention. In various aspects, the pharmaceutical spray composition is a unit dose formulation. In various aspects the pharmaceutical spray composition further comprises polyacrylic acid, poloxamer 188 or PEG.

The compositions of the invention may be formulated for administration using a spray-on system. In one exemplary spray system, the nanoparticles within the composition may or may not be suspended or dissolved in a carrier such as water. In another spray system, The nanoparticles within the compositions are suspended or dissolved at various ratios in a water miscible such as DMSO, NMP, dimethylformamide (DMF) or tetrahydrofuran (THF). The compositions are then administered directly on the internal or external site of injury using a spray system, a brush system or syringe-type system. The spray system may be an aerosol spray or electrostatic spray. Alternatively, these compositions may be introduced to the injury using an endoscopic or other laproscopic device.

The disclosure provides pharmaceutical spray compositions formulated for delivery of nanoparticles at 1 mg/kg to 1 g/kg, 10 mg/kg to 1 g/kg, 20 mg/kg to 1 g/kg, 30 mg/kg to 1 g/kg, 40 mg/kg to 1 g/kg, 50 mg/kg to 1 g/kg, 60 mg/kg to 1 g/kg, 70 mg/kg to 1 g/kg, 80 mg/kg to 1 g/kg, 90 mg/kg to 1 g/kg, 10 mg/kg to 900 mg/kg, 10 mg/kg to 800 m/kg, 10 mg/kg to 700 mg/kg, 10 mg/kg to 600 mg/kg, 10 mg/kg to 500 mg/kg, 10 mg/kg to 400 mg/kg, 10 mg/kg to 300 mg/kg, 10 mg/kg to 200 mg/kg, 10 mg/kg to 100 mg/kg, 10 mg/kg to 75 mg/kg, 10 mg/kg to 50 mg/kg, 50 mg/kg to 900 mg/kg, 100 mg/kg to 800 mg/kg, 200 mg/kg to 700 mg/kg, 300 mg/kg to 600 mg/kg, 400 mg/kg to 500 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, or more.

Single dose administrations are provided, as well as multiple dose administrations. Multiple dose administration includes those wherein a second dose is administered within minutes, hours, day, weeks, or months after an initial administration.

### Uses of the Compositions

A method of treating a condition in an individual is provided comprising the step of administering the spray compositions of the disclosure to a patient in need thereof in an amount effective to treat the condition. In various aspects, the individual has a bleeding disorder. Methods are provided wherein the spray composition is administered in an amount effective to reduce bleeding time by more than 15%, by more than 20%, by more than 25%, or by more than 30% compared to no administration or administration of saline. In various aspects, the method is used wherein the bleeding disorder is a symptom of a clotting disorder, an acquired platelet function defect, a congenital platelet function defect, a congenital protein C or S deficiency, disseminated intravascular coagulation (DIC), Factor II deficiency, Factor V deficiency, Factor VII deficiency, Factor X deficiency, Factor XII deficiency, Hemophilia A, Hemophilia B, Idiopathic thrombocytopenic purpura (ITP), von Willebrand's disease (types I, II, and III), megakaryocyte/platelet deficiency. In various aspects, a method is provided wherein the condition is thrombocytopenia arising from chemotherapy and other therapy with a variety of drugs, radiation therapy, surgery, accidental blood loss, and other specific disease conditions. In various aspects, a method is provided wherein the condition is aplastic anemia, idiopathic or immune thrombocytopenia (ITP), including idiopathic thrombocytopenic purpura associated with breast cancer metastatic tumors which result in thrombocytopenia, systemic lupus erythematosus, including neonatal lupus syndrome, metastatic tumors which result in thrombocytopenia, splenomegaly, Fanconi's syndrome, vitamin B 12 deficiency, folic acid deficiency, May-Hegglin anomaly, Wiskott-Aldrich syndrome, paroxysmal nocturnal hemoglobinuria, HIV associated ITP and HIV-related thrombotic thrombocytopenic purpura; chronic liver disease; myelodysplastic syndrome associated with thrombocytopenia; paroxysmal nocturnal hemoglobinuria, acute profound thrombocytopenia following C7E3 Fab (Abciximab) therapy; alloimmune thrombocytopenia, including maternal alloimmune thrombocytopenia; thrombocytopenia associated with antiphospholipid antibodies and thrombosis; autoimmune thrombocytopenia; drug-induced immune thrombocytopenia, including carboplatin-induced thrombocytopenia, heparin-induced thrombocytopenia; fetal thrombocytopenia; gestational thrombocytopenia; Hughes' syndrome; lupoid thrombocytopenia; accidental and/or massive blood loss; myeloproliferative disorders; thrombocytopenia in patients with malignancies; thrombotic thrombocytopenia purpura, including thrombotic microangiopathy manifesting as thrombotic thrombocytopenic purpura/hemolytic uremic syndrome in cancer patients; autoimmune hemolytic anemia; occult jejunal diverticulum perforation; pure red cell aplasia; autoimmune thrombocytopenia; nephropathia epidemica; rifampicin-associated acute renal failure; Paris-Trousseau thrombocytopenia; neonatal alloimmune thrombocytopenia; paroxysmal nocturnal hemoglobinuria; hematologic changes in stomach cancer; hemolytic uremic syndromes in childhood; and hematologic manifestations related to viral infection including hepatitis A virus and CMV-associated thrombocytopenia. In various aspects, a method is provided wherein the condition arises from treatment for AIDS which result in thrombocytopenia. In various aspects, the treatment for AIDS is administration of AZT.

In various aspect, the individual being treated is suffering from a wound healing disorders, trauma, blast trauma, a spinal cord injury, hemorrhagic stroke, hemorrhaging following administration of TPA, or intraventricular hemorrhaging which is seen in many conditions but especially acute in premature births.

### Porcine Liver Trauma Model

Clinical translation of any intravenous hemostat requires both scaling material synthesis and the investigation of safety and efficacy in larger species. While at a molecular level, hemostasis appears to be well-conserved, there is a significant difference in hemodynamics and blood coagulation parameters that may not be fully conserved from rodents to humans (Siller-Matula et al., Thromb. Haemost. 100: 397-404 (2008). Porcine hemorrhagic injury models have been developed for vascular trauma (femoral vessels) (Johnson et al., US Army Med. Depart. J. 36-39 (2012), Gegel et al., US Army Med. Depart. J. 31-35 (2012)), solid organ injury (liver, spleen) (Velmahos et al., Am. Surg. 74: 297-301 (2008), Gurney et al., J. Trauma, 57: 726-38 (2004)), thoracic injury (lung) (Baker et al., Crit. Care Med. 40: 2376-84 (2012), and polytrauma (solid organ/femur). 18-20 Pigs are often used as a preclinical model of uncontrolled hemorrhage, as their hemodynamics and size are relatively well-scaled to humans (Siller-Matula et al., Thromb. Haemost. 100: 397-404 (2008).

The pig is the standard model for uncontrolled hemorrhagic trauma, when investigating the physiological impact of a potential therapy. The cardiovascular system is well-correlated with human parameters and the comparable size allows for devices to be used in both clinical and research environment without modification. Furthermore, the wound-healing process appears to be similar to humans, resulting from similarities between porcine and human skin.

The use of intravenous hemostatic agents has been shown to reduce bleeding times both in vitro and in vivo (rat), as well as lead to significant increases in survival after a lethal liver trauma in rats (Bertram et al., Sci. Translational Med. 1: 11ra22 (2009)). In order to address the difference in hemodynamics between small and large animal, the efficacy of the hemostatic nanoparticles in a large animal, porcine model of hemorrhage was studied. In Example 1, the use of intravenous hemostatic nanoparticles to reduce blood loss and increase survival after a solid organ injury was examined.

It was determined that the administration of the nanoparticles may induce CARPA, a pseudoallergy that has just recently begun to be characterized, and appears to be elicited readily in pigs.

Experiments in the naïve pig model have shown that the excipient poly(acrylic acid) alone is not responsible for initiating the CARPA response, as the injection of neutrally charged particles (+PAA) did not itself induce a response. However, the pig in the experiment showed a more severe reaction to the particles with the PAA. While it is possible, that the PAA is directly responsible for increasing the severity, it is also likely that the response was increased due to an already heightened and active complement system. Subclinical reactions to PAA may still exist.

Szebeni et al. have postulated that zeta potential is one potential mediator of CARPA induced by intravenous nanoparticle systems (Szebeni et al., Nanomedicine, 8: 176-84 (2012)). While the mechanism is currently not fully understood, both the results presented by Szebeni et al. and studies with the neutral particles, suggest that neutrally charged nanoparticles may mitigate the initiation of CARPA in pigs. Additional research is needed to elucidate this mechanism so that the parameters to minimize CARPA may be identified.

The mechanism of CARPA and its relation to the coagulation cascade have not yet been fully elucidated. However, there are prior indications that biomaterials in contact with blood have the potential to elicit complement activation, which are mediated by FXII activation, and its fragments (factor XI1) Charged, or hydrophilic materials, tend to adsorb proteins and produce FXII fragments as well as kallikrein (which in turn cause bradykinin formation—a strong vasodilator).

If CARPA is indeed mediated by factor XII activation by adsorption to the charged nanoparticle surface, then its fragments may well induce coagulopathy by activating plasminogen, and further cause additional hemorrhage due to bradykinin (or histamine) vasodilation. While long-term coagulopathy was not observed clotting time and APTT assays, it is possible that this coagulopathy is transient, and only catastrophic when occurring simultaneously with an injury.

### Mitigation of Response of CARPA

Diphenhydramine, phenylephrine, epinephrine and steroids may also be used in conjunction to reverse the anaphylaxis induced by CARPA (Johnson et al., J. Pharma. Sci. 100: 2685-92 (2011)). Unfortunately for the application of intravenous hemostatic agents to be administered during trauma, co-administration with additional pharmaceuticals should be avoided if possible.

One potential method for reducing the onset of CARPA is to infuse the nanoparticles slowly (or with multiple small doses) (tachyphylaxis) (Szebeni et al., Nanomedicine, 8: 176-84 (2012)). This appeared to prevent the onset of CARPA and reduce the severity of any symptoms. It relies on a desensitization mechanism. However, since the present therapy will rely on rapid administration after hemorrhagic injury, tachyphylaxis does not appear to be a viable option.

The most viable option for prevention of CARPA appears to be tuning the zeta potential of the targeted nanoparticles to be close to neutral. The GRGDS (SEQ ID NO: 2) targeting ligand is inherently negatively charged due to the presence of Arg (+), Asp (−) and the carboxylic acid terminus (−). One potential mitigation for this study is to substitute the GRGDS (SEQ ID NO: 2) targeting peptide for one with a neutral charge, such as a cyclic RGD, which has both a higher specificity for activated platelet GPIIb/IIIa and a net neutral charge.

The experiments described herein demonstrate that CARPA induced by nanoparticle administration produces massive hemorrhage when administered during a large hemorrhagic injury. Coagulopathy may still be present, even after an episode of CARPA (characterized by cardiopulmonary dysfunction) has passed. However, this response is transient and can be modulated by tuning the parameters of intravenous hemostatic nanoparticles, specifically by neutralizing their charge (zeta potential).

### EXAMPLES

### Example 1

### Nanoparticle Synthesis

Nanoparticles were synthesized from poly (lactic-co-glycolic acid)-poly-_{L}-lysine (PLGA-PLL) block copolymer conjugated with polyethylene glycol (PEG) arms. Spherical nanoparticles were fabricated using a nano precipitation method as described herein. Dexamethasone was dissolved in a solvent, and the appropriate amount of polymer was also dissolved and mixed with the drug. The drug/polymer solution was pipetted dropwise into spinning 1×PBS. The resultant solution was allowed to stir uncovered for approximately 20 min at room temperature. After the nanospheres stir hardened, the pH was adjusted down to 3.0-2.7 to induce flocculation. This pH range was found to be useful for flocculation to occur. The nanospheres were purified by centrifugation (500 g, 3 min, 3×), resuspended in deionized water, frozen, and freeze-dried on a lyophilizer. A release study was performed by dissolving 10 mg of nanospheres into 1 mL 1×PBS, repeated in triplicate.

Size of the nanospheres was determined by dynamic light scattering (DLS). Conformation of size and morphology was determined by a scanning electron microscope (SEM). The amount of drug was determined by dissolving spheres in DMSO and running on a UV-Vis. Release study data was gathered at various time points and was run on UV-Vis to determine how dexamethasone elutes out of the nanoparticles over time.

### Example 2

### Attachment of Peptides to Nanoparticles

The yield and time to make product has been significantly reduced by determining the shortest times necessary for intermediate treatment steps. Yield is significantly increased using centrifugation to collect PLGA-PLL-PEG after precipitating. Yield is also significantly increased with nanoprecipitation nanoparticle formation method and even further increased if using the poly(acrylic acid) coacervate precipitation technique for nanoparticle collection.

Once the PLGA-PLL-PEG is synthesized, the active peptide such as GRGDS (SEQ ID NO: 2) needs to be coupled to the polymer.

When the quad block polymer (PLGA-PLL-PEG-peptide) was used, yield of spheres was extremely low. Since the peptide was the most expensive portion of the polymer, a method was employed to form spheres from the triblock (PLGA-PLL-PEG) and then attach the peptide to the spheres themselves.

Conjugation of the peptide to triblock nanoparticles led to approx. 50% conjugation efficiency (calculated as the arginine to lysine ratio).

However, it was found that an extra rinse step of the nanospheres before amino acid analysis led to significant loss of the peptide with a conjugation efficiency of 11%. Upon scaling the reaction up for a 1 g batch of nanospheres, the conjugation efficiency essentially dropped to 0%. Therefore, a method was pursued that would allow one to make the entire quad block polymer and with at least comparable yield produce nanoparticles with a tight size distribution.

This approach led to the manufacture of a quadblock polymer prior to the formation of the nanoparticle. The quadblock conjugation efficiency was approximately 80%, but dropped to 13% after nanosphere formation using the nanoprecipitation technique with and without poly(acrylic acid). Finally, the quadblock was made by reactivating the polymer with CDI in DMSO immediately prior to the addition of the peptide. This step increases the conjugation of peptide to above 50% (n=3).

### Emulsion Method

The emulsion method succeeds in making spheres of diameter between 326-361 nm.

The emulsion method stir-hardens the nanospheres in 50 ml of 5% PVA in deionized water. Scaling up the production of nanospheres using this method requires large volumes of solution for stir hardening. This observation, coupled with the fact that prior methods added the peptide for the conjugation step after forming the particles, means that a very large amount of peptide would be needed for the large volume of solution to achieve a reasonable coupling efficiency.

For the nanoprecipitation method, scaled down version, stir hardening in 10 ml PBS was carried out with simultaneous conjugation of the peptide. This step adds a sufficient amount of peptide. The nanoprecipitation method also lends itself to the formation of nanoparticles with the quadblock polymer eliminating the need for a post-fabrication coupling reaction.

There are a number of fundamental issues identified with nanoparticles, including uniformity of particles, aggregation of particles, challenges in resuspending nanoparticles and challenges of resuspending following lyophilization

Groups have come up with a number of approaches to deal with these challenges. For example, one can have a lyoprotectant to resuspend small nanoparticles following lyophilization. (Sauaia et al., J. Trauma 38: 185 (1995), Champion et al., J. Trauma 54: S13 (2003)). Other found that through nanoprecipitation technique coupled with the use of poly(acrylic acid) to flocculate the particles, the need to add a lyoprotectant to the solution was avoided.

### Nanoprecipitation

The nanoprecipitation method uses dropwise addition of polymer dissolved in a water miscible solvent such as acetonitrile to make spheres of consistent size (Regel et al., Acta. Anaesthesiol. Scand. Suppl 110: 71 (1997); Lee et al., Exp. Opin. Investig. Drugs 9: 457 (2000); Blajchman, Nat. Med. 5: 17 (1999); Lee et al., Br. J. Haematol. 114: 496 (2001)).

### Poly(Acrylic Acid) Coacervate Precipitation

This method modified from (Regel et al. (1997); Kim et al., Artif. Cells Blood Substit. Immobil. Biotechnol. 34: 537 (2006)) was employed to increase yield of nanoparticles and to reduce aggregation of spheres during centrifugation and lyophilization steps as had previously been observed. The precipitation allows for gentle centrifugation <500 g.

The size reproducibility has thus far been shown to be an advantage over the emulsion and nanoprecipitation alone methods which is highly dependent on sonication conditions to make a homogenous size distribution. SEM image shows morphology of nanoparticles and homogeneity of size. Histogram inlay was made from 100 measurements of nanoparticle diameter, and shows size distribution is centered around 236.1 nm+/−56.6 nm.

### Method for Making PAA-Coated Nanoprecipitated Synthetic Platelets

PLGA (Resomer 503H) was purchased from Evonik Industries. Poly-1-lysine and PEG (˜4600 Da MW) were purchased from Sigma Aldrich. All reagents were ACS grade and were purchased from Fisher Scientific. PLGA-PLL-PEG coblock polymer was made using standard bioconjugation techniques as previously described (Lavik et al).

### Quadblock Conjugation

PLGA-PLL-PEG was dissolved in N-methyl-2-pyrrolidone (NMP) to a concentration of 100 mg/ml. Two molar equivalents of CDI were added to reactivate the PEG groups and stirred for 1 hour. Twenty five mg of oligopeptides (GRGDS (SEQ ID NO: 2) was dissolved in 1 ml NMP and added to the stirring polymer solution. This mixture was reacted for 3 hours, and then transferred to dialysis tubing (SpectraPor 2 kDa MWCO). Dialysis water was changed every half hour for 4 hours with Type I D.I. water. The product was then snap-frozen in liquid nitrogen and lyophilized for 2 days.

### Nanoprecipitation

The resulting quadblock copolymer PLGA-PLL-PEG-GRGDS was then dissolved to a concentration of 20 mg/ml in acetonitrile. This solution was added dropwise to a stirring volume of PBS. The general rule is to use twice the volume of PBS as acetonitrile. Precipitated nanoparticles formed as the water-miscible solvent dissipates. However, to scale up to quantities greater than 300 mg starting quadblock, it was found that priming the precipitation volume with acetonitrile reduced the spontaneous formation of aggregates. Solvent:water ratios were adjusted throughout the precipitation process so that the final concentration in the precipitation volume is 2:1 PBS:acetonitrile. The particles were then stir-hardened for 3 hours. Particles were then collected using centrifugation @ 15000 g and rinsing with PBS 3 times. Alternatively, particles were collected using the coacervate precipitation method.

### Coacervate Precipitation

One mass equivalent of dry poly(acrylic acid) was added to the stirring particle suspension. 1% w/v pAA was then added to the stirring suspension until flocculation occurs. Stirring was paused momentarily after each addition of pAA to observe flocculation. After 5 minutes, the flocculated particles were collected by centrifugation at 500 g, and rinsed 3 times with 1% pAA (centrifuging @ 500 g, 2 m, 4 C between rinses). On the final rinse, particles were resuspended with D.I. water, snap-frozen and lyophilized for 2-5 days, depending on the final volume of water.

### Resuspension

Particles were massed and resuspended to a concentration of 20 mg/ml in 1×PBS. Particles are either vortexed to resuspend, or alternatively vortexed and briefly sonicated at 4 W to a total energy of 50 J using a probe sonicator (VCX-130, Sonics & Materials, Inc.).

### Example 3

### In Vivo Testing in the Femoral Artery Injury Model

In preliminary work, a femoral artery injury model was used. It is a very clean model that allows simple assessment of the impact of a therapy on bleeding. Male Sprague-Dawley rats were anesthetized with isoflurane. The animal's temperature was maintained using a heating pad and monitored throughout the experiment using a temperature probe. An arterial catheter was used for measuring blood pressure and blood draws, and a venous catheter was used for administration of the agent being tested. The abdominal cavity was opened, and the median lobe of the liver is cut sharply 1.3 cm from the superior vena cava following. The cavity was immediately closed, and the experimental agent was delivered.

Blood samples were drawn immediately before the injury, at 5 minutes post injury, and at 30 minutes post injury. Animals were maintained for 60 minutes or until death. At the end of 60 minutes, pre-weighed sponges were used to collect the blood in the abdominal cavity to determine blood loss. All the major organs were collected for histology and biodistribution of the nanoparticles.

Nanoparticles of the invention were intravenously administered into a canulated femoral vein in 0.5 ml injection volume (20 mg·ml), 3 minute injections with 5 minute equilibration shortly after injury. The nanoparticles administered had a PLGA-PLL nanosphere core (˜200 nm), multiple 4600 kD PEG arms and one of the following RGD peptides conjugated to the PEG arms: RGD, RGDS (SEQ ID NO: 1), and GRGDS (SEQ ID NO: 2).

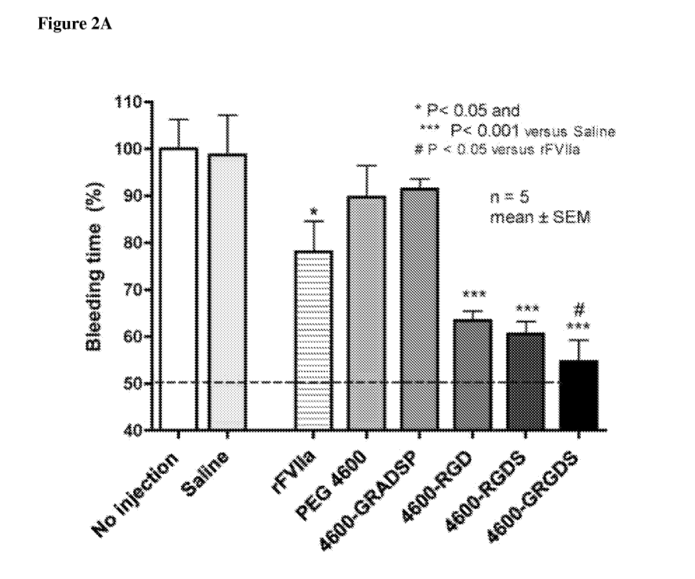

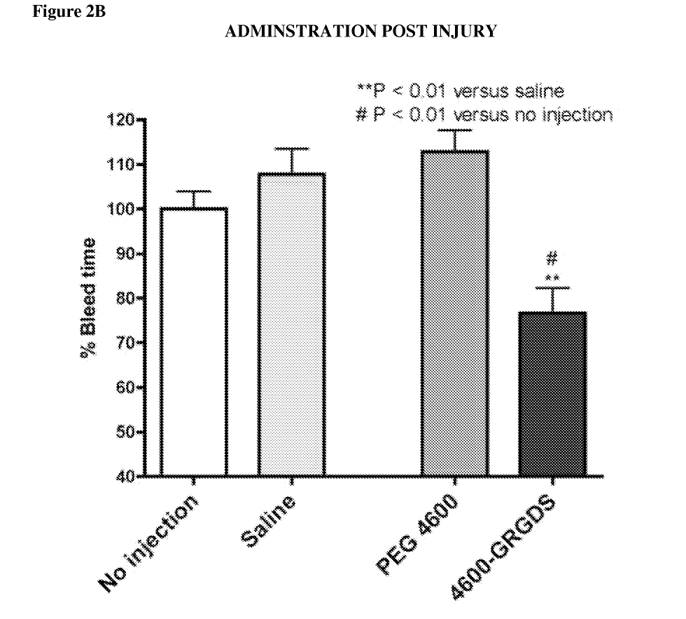

The effect these nanoparticles had on bleeding time was compared to saline control, recombinant Factor VIIa and nanoparticles which comprised PEG alone. All of the nanoparticles comprising a RGD peptide significantly reduced bleeding time. The nanoparticles were either administered immediately prior to injury (see FIG. 2A) or post-injury (see FIG. 2B). When administered post-injury, the nanoparticle comprising the 4600-GRGDS peptide significantly reduced % bleed time compared to nanoparticles only comprising PEG (PEG 4600). (See FIG. 2B)

### Example 4

### Porcine Liver Trauma Model

### Liver Resection Model

Animal protocols were developed based on Gurney et al. 16, and were adapted in conjunction with the Trauma Research Laboratory at Massachusetts General Hospital, and approved by the Case Western Reserve University IACUC. The goal of the liver injury study was to determine safe and efficacious dose levels of the nanoparticle treatment. The initial dose was started at roughly 20 mg/kg and dosed down by a factor of 10 until a safe dosage was reached, followed by a factor of 2 until no effect was observed (−0.03 mg/kg).

Yorkshire pigs (30-35 kg) were anesthetized with telazol (6-8 mg/kg i.m.), intubated, placed on a ventilator, and maintained on isoflurane (2-2.5%). Catheters were placed in the carotid artery for arterial sampling and invasive blood pressure monitoring, as well as in the internal jugular vein for drug administration and saline infusions. A laparotomy was performed, and the left lobe of the liver isolated from the underlying anatomy with a malleable retractor. This provides a collection surface for suctioning blood, after injury. The left lobe was resected 2″ from the apex (measured from the most distal part of the lobe) with a #15 scalpel blade. Treatments were administered i.v. 5 minutes after the injury was created, and consisted of active intravenous hemostat (GRGDS-NP (SEQ ID NO: 6)), scrambled particles (Scrambled-NP) and saline (lactated ringers).

Blood loss was measured directly by suctioning blood immediately from the abdominal cavity, but maintaining a sweep radius of approximately 1 cm to prevent removal of clot from the injury surface. Arterial blood samples were collected at baseline, 15, 30, 60, 120, 180, and 240 minutes after injury, and were immediately followed by lactated ringers infusions: 400 ml @ 40 ml/min for the first time point (15 min) and 200 ml @ 20 ml/min for all subsequent time points that the MAP is below baseline.

Outcomes considered include physiological parameters: heart rate (HR), mean arterial pressure (MAP), Sp0₂, and ETCO₂. Blood samples are analyzed for platelet counts, blood gas, and diagnostic clotting times (ROTEM and Hemochron). The animal was monitored for 4 hours after injury or death, at which point pigs were euthanized with an overdose of sodium pentobarbital.

### Naïve Administration/Response Model

The initial results with this pig model indicated an adverse impact of the experimental nanoparticle therapeutic when dosed higher than 0.15 mg/kg. This adverse response was characterized by rapid hemorrhage from the induced liver injury. A naive administration model was developed to determine the impact of the nanoparticles in the absence of an injury. Here, the formulation of the nanoparticles was varied to look at the influence of 2 factors: excipient (+/−polyacrylic acid), and zeta potential (−30 mV, neutral, and +20 mV).

The surgery was performed as described above to introduce catheters for invasive blood pressure monitoring, arterial blood sampling and venous infusions. A dose of 2 mg/kg of nanoparticles was injected, denoting time=0. The pig was then monitored for 1 hour, with regards to physiological parameters: heart rate (HR), mean arterial pressure (MAP), Sp0₂, and ETCO₂. Blood samples were analyzed for platelet counts, blood gas, and diagnostic clotting times (ROTEM and Hemochron).

After 1 hour, a second formulation of the nanoparticles was injected, and the naive administration model experiment repeated. N=2 pigs were used in this experiment. The first pig received 2 doses of PLA-PEG-NP's (zeta=−30 mV) with (t=0 min) and without the PAA excipient (t=85 min). The second pig received 2 doses of PLGA-PEG-NP's (with PAA), comparing zeta potentials of −1.29 mV (t=0) and +20 mV (t=65 min).

### Making a Reproducible Model

Creating a reproducible liver injury was crucial to producing a consistent injury model. The initial, and only criteria, during our initial experiments is that we resect the left lobe of the liver, measuring 2″ from the apex. When comparing the blood loss in the pre-administration time (0-5 minutes), it was observed that there was a very large variation between pigs. This was reduced to a consistent 300-400 ml, after the liver injury was standardized as described. This was primarily achieved by establishing a consistent degree of injury as well as the angle of the cut, measuring 2″ from the left lobe apex, and ensuring that measurements were equivalent. Replacement of the injured left lobe in its natural resting place, was also critical to prevent tension/torsion from altering normal hepatic blood flow. Ring clamps were held in place placed during the injury, and proximal, to maintain consistency with the previously established injury protocol (Gurney et al., J. Trauma 57: 726-38 (2004).

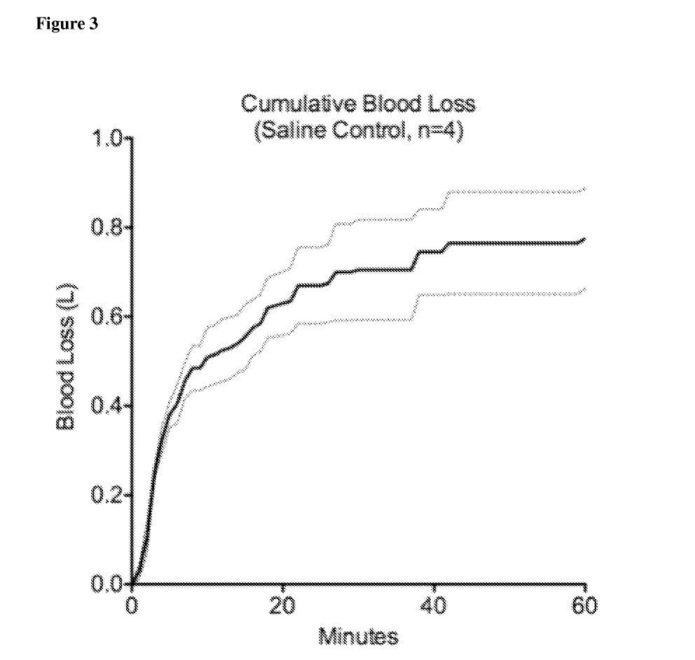

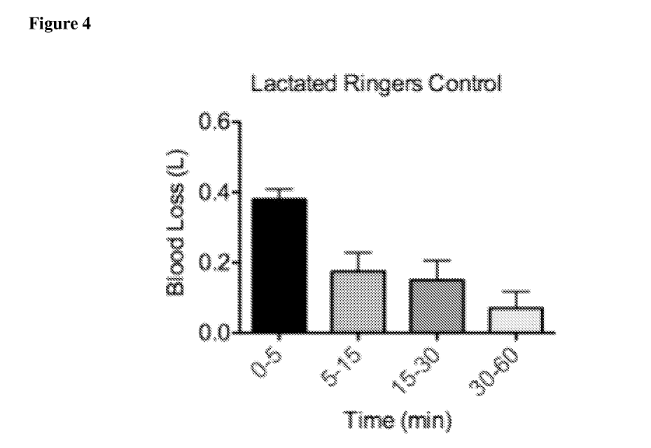

In our initial work, the pre-administration blood loss (0-5 minutes) was highly variable, indicating an irreproducible injury model. This was later ameliorated by tightly standardizing the injury. The comparison of cumulative blood loss (FIG. 3), or blood loss at relevant experimental times points (FIG. 4) before and after particle administration appears to be one metric that may be able to be used to measure hemostatic efficacy of these particles, and minimize the impact of the disparity in pre-administration blood loss between pigs.

### Example 5

### Air Brush System

A 2% solution of the polymer in NMP (wt/vol) was made as described in Example 1. An airbrush spray system was used to spray the solution onto the exposed surface of the liver following the left lobe liver resection in a pig.

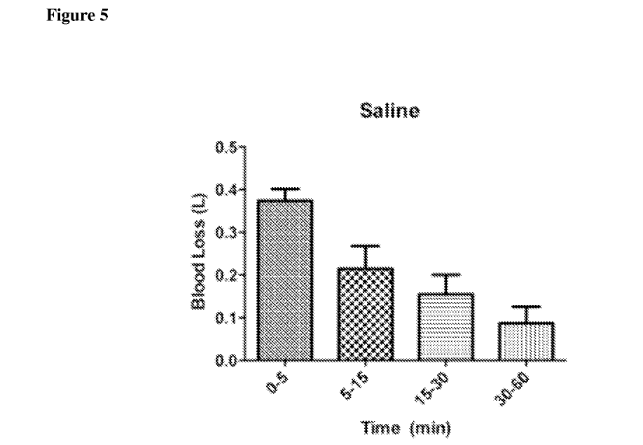

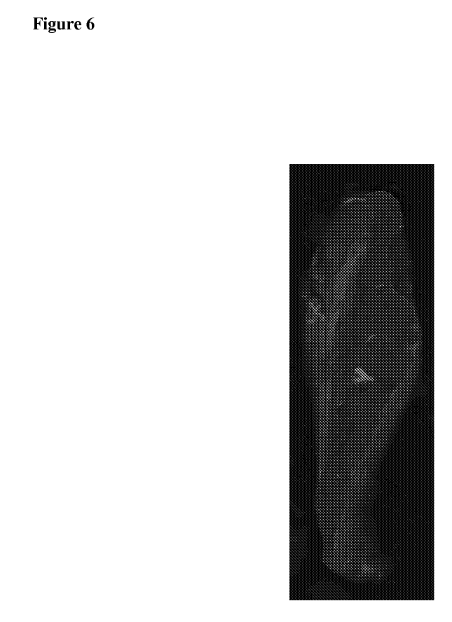

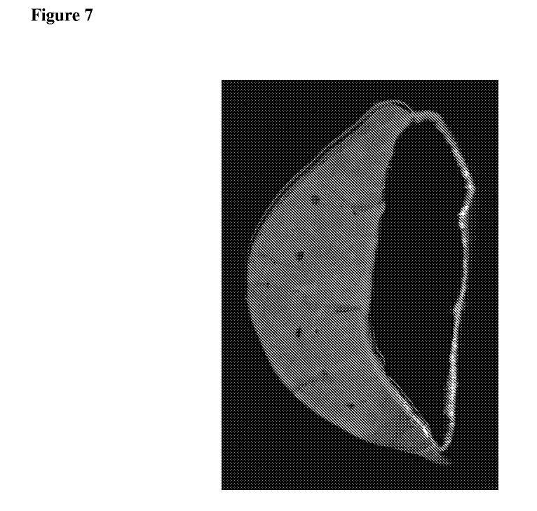

Generally in this resection, the pig bleeds over the course of several hours. The first hour is summarized below and in FIG. 5. When the spray-on system was administered to the exposed surface of the liver, it formed a film across the liver that immediately and completely stopped the bleeding. FIG. 6 shows the liver and the trapped blood. The animal survived with no more blood loss to the end of the 4 hour experiment. It was stable with a solid heart rate, CO₂, blood pressure, and temperature. The liver injury exposes several major vessels as can be seen in the section of the liver that was actually removed (FIG. 7).

This formulation works over a very wide concentration from 0.1% polymer to, potentially, as high as 99% polymer. Multiple administration systems are used to deliver it to the surface including spray systems, brush systems and syringe-type systems. Alternatively, the compositions may be administered to the subject using an endoscope or other laproscopic device. Finally, the compositions of the invention may be administered via catheter.

The full PLGA-PLL_PEG-GRGDS polymer is not necessary to have the effect observed above. Among other variations, and without limitation, PLA could be substituted, PLL could be left out, PEG of different lengths could be used, and the RGD may or may not be critical depending on the formulation.

A control experiment in which only the delivery solvent, NMP, was delivered in the liver injury model does not stop the bleeding. However, the airbrush system is extremely effective sealing the wound essentially shortly after administration and the wound remained sealed for the duration of the experiment (1 hour before termination in the case of the data in FIG. 5).

### Example 6

### Evaluation of Spray Devices

Various types of spraying devices were investigated to determine whether the type of device may be used to apply the compositions of the invention and to measure the efficiency of the application device on the hemostatic system. A standard hand sprayer was used to apply a 5% solution (w/v) of the polymer (PLGA-PLL-PEG-GRGDS, “quad polymer”) or control polymer (PLGA 503H) in the delivery solvent NMP. Bleeding in liver injury model in rats following median lobe resection was treated with the polymer solution administered using the standard sprayer. The results of this study are provided in Table 1.

TABLE 1

| | Survival Time | |
|---|---|---|
| Treatment | (Minutes) | blood ml/kg |
| quad | 12 | 25.58838475 |
| quad | sac (1 hr) | 9.098909657 |
| quad | 9 | 20.92980769 |
| quad | 8 | 18.5631016 |
| 503H | 41 | 12.67909483 |
| 503H | 10 | 33.39158163 |

The PLGA-PLL-PEG-GRGDS nanoparticles stopped bleeding and improved survival when properly applied. However, when the nanoparticles were applied using the hand spray bottle, application was difficult, and the formation of a complete film across the wound varied. Only 1 animal (in the treatment group; denoted with * in Table 1) survived the whole time, and survival was directly related to the film formation. One animal in the control (503H) group survived to 41 minutes, but the film began to detach from the tissue and bleeding commenced leading to more blood loss and death at the 41 minute time point. Therefore, different spray bottles and different concentrations of polymer in solution were tested on chicken breasts (purchased as a grocery store).

### Sprayer #1

The first sprayer tested was a vintage style refillable empty glass perfume bottle with an spray atomizer (1.64 oz). The solution tested was 503H PLGA in n-methyl-2-pyrollidone (NMP). The polymer solution was sprayed onto the chicken breast from about 5 inches away. The data is provided in Table 2.

TABLE 2

| | # | Film | Time Given | | |
|---|---|---|---|---|---|
| Concentration | Sprays | Formation? | to Form | | Notes |
| 10 mg/mL | 7-8 | No | 15 | min | No film formed |
| 20 mg/mL | 7-8 | No | 12 | min | No film formed |
| 30 mg/mL | 7-8 | No | 5 | min | No film formed |
| 100 mg/mL | 8 | Yes* | 5 | min | Film formed but |
| | | | | | not strong enough |
| | | | | | to peel away |
| 100 mg/mL | 12 | Yes | 5 | min | Film formed and |
| | | | | | was able to peel |
| | | | | | away from |
| | | | | | chicken breast |
| 100 mg/mL | 16 | Yes | 5 | min | Film formed and |
| | | | | | was able to peel |
| | | | | | away from |
| | | | | | chicken breast |
| 100 mg/mL | 14 | Yes* | 15 | seconds | Wet film formed |
| | | | | | and clumped |
| | | | | | when pulled off |
| 100 mg/mL | 14 | Yes* | 30 | seconds | Wet film formed |
| | | | | | and clumped |
| | | | | | when pulled off |

| | | | | |
|---|---|---|---|---|
| 200 mg/mL | 8 | No | 0, Instant | No film instantly |
| 200 mg/mL | 20 | No | 0, Instant | No film instantly |

### Sprayer #2

The second sprayer tested was a funnel shaped black atomizer (5 ml). The solution tested was 503H PLGA in n-methyl-2-pyrollidone (NMP). The polymer solution was sprayed onto the chicken breast from about 5 inches away. The data is provided in Table 3.

TABLE 3

| | # | Film | Time Given | |
|---|---|---|---|---|
| Concentration | Sprays | Formation | to Form | Notes |
| 100 mg/mL | 6 | Yes* | 30 seconds | Film formed but |
| | | | | was unable to |
| | | | | retrieve from |
| | | | | chicken in |
| | | | | one piece |
| 100 mg/mL | 4 | Yes* | 30 seconds | Film formed but |
| | | | | was wet and |
| | | | | clumped when |
| | | | | pulled off |
| 200 mg/mL | 5 | Yes | 0, Instant | Was able to get |
| | | | | a film pulled |
| | | | | of instantly |
| 200 mg/mL | 6 | Yes* | 0, Instant | Was able to pull |
| | | | | off a film but |
| | | | | was very wet |
| 200 mg/mL | 4 | Yes | Instant | Was able to pull |
| | | | | off a film but |
| | | | | was very wet |
| | | | | *possible misfire/ |
| | | | | clogging |

### Sprayer #3

The third sprayer tested was a refillable perfume atomizer shaped black atomizer (7 ml). The solution tested was 503H PLGA in n-methyl-2-pyrollidone (NMP). The polymer solution was sprayed onto the chicken breast from about 5 inches away. This atomizer frequently clogged, was unable to spray 100 mg/ml solution and did not allow the solution to be changed easily.

### Conclusion

Sprayer #2 is most suitable for application of spray compositions of the invention as it delivers the highest volume in the fewest number of sprays and is able to administer more polymer solution in less time. The solution of 200 mg/mL nanoparticles provided the best film formation of those tested.

Based on these findings, the rat study was repeated the rat liver injury model using sprayer #2 and a solution of 200 mg/ml. The data for this study is provided in Table 4.

TABLE 4

| | Survival Time | |
|---|---|---|
| Treatment | (Minutes) | blood ml/kg |
| 503H | sacrificed | 19.97866287 |
| 503H | 9 | 21.8144 |
| 503H | Sacrificed | 0 |
| quad | 7 | 24.68451243 |

Two of the PLGA 503H animals survived for the duration of the experiment and the animals were sacrificed after an hour. Similar to the rat study, the observations from the surgeries and application demonstrated that a complete film was critical for success of the nanoparticle treatment and the variation in force of the sprayer directly impacted the film formation.

Based on these studies, the quad polymer adheres better to the tissue than the PLGA control and a sprayer that provides a strong, uniform application is critical for initially sealing the wound. Varying the polymer to solvent ratio from 2% to 20% demonstrated that the only limitation on the concentration appears to be the sprayer system and what can be moved through the apparatus.

### Example 6

### Nanoparticle Administration Exacerbates Bleeding

Nanoparticle compositions NP1 and NP100 were administered. NP100 refers to a formulation with approximately 100 times as much peptide on the surface as the NP1 formulation. Administration of the nanoparticles caused an unexpected, massive bleed-out at doses >=2 mg/kg, independent of the peptide attached. This occurred with the NP100 and NP1 particles (varying peptide density), and it occurred regardless of the peptide attached (GRGDS (SEQ ID NO: 2), GRADSP (SEQ ID NO: 3), or none). This is readily seen in survival time, and total blood loss, where control groups given lactated ringers (n=4/4) survived the entire duration of the 240 minute experiment, with a mean 775 ml blood loss+/−225 S.D., whereas the particle treatment groups faired considerably worse (Table 5).

Table 5 provides survival time and blood loss grouped by dose (mg/kg). All 4/4 lactated ringers control pigs survived the entire 240 minutes, with a mean blood loss of 775 ml+/−225 S.D. The optimal dosing appears to be between 0.1-0.2 mg/kg, where the adverse impact appears to be minimized. Interestingly, dosing down to 0.03 mg/kg, appears to also exacerbate the injury model, however, not as drastically as was observed with doses >2.0 mg/kg. Rather, animals are susceptible to prolonged bleeding times instead of induction of rapid hemorrhage.

TABLE 5

| | Survival Time (min) | | | Blood Loss (ml) | | |
|---|---|---|---|---|---|---|
| Dose (mg/kg) | Mean | S.D. | N | Mean | S.D. | N |
| Saline | | | | | | |
| Control | 240 | 0 | 4 | 775 | 224.7 | 4 |
| NP1 | | | | | | |
| Scrambled | | | | | | |
| 0.03 | 210 | | 1 | 1260 | | 1 |
| 0.10 | 26 | 28.3 | 3 | 920 | 408.4 | 3 |
| 0.20 | 7 | | 1 | 880 | | 1 |
| 2.00 | 8 | | 1 | 1040 | | 1 |
| GRGDS | | | | | | |
| (SEQ ID NO: 2) | | | | | | |
| 0.03 | 30 | | 1 | 1240 | | 1 |
| 0.10 | 144 | 93.1 | 3 | 853 | 391.1 | 3 |
| 0.20 | 240 | | 1 | 1020 | | 1 |
| 2.00 | 9 | 0.0 | 2 | 890 | 14.1 | 2 |
| NP100 | | | | | | |
| Scrambled | | | | | | |
| 0.10 | 73 | 77.6 | 5 | 1335 | 168.6 | 5 |
| 0.20 | 87 | | 1 | 820 | | 1 |
| GRGDS | | | | | | |
| (SEQ ID NO: 2) | | | | | | |
| 0.10 | 172 | 81.4 | 6 | 1086 | 545.6 | 6 |
| 0.20 | 87 | 132.2 | 3 | 992 | 246.0 | 3 |

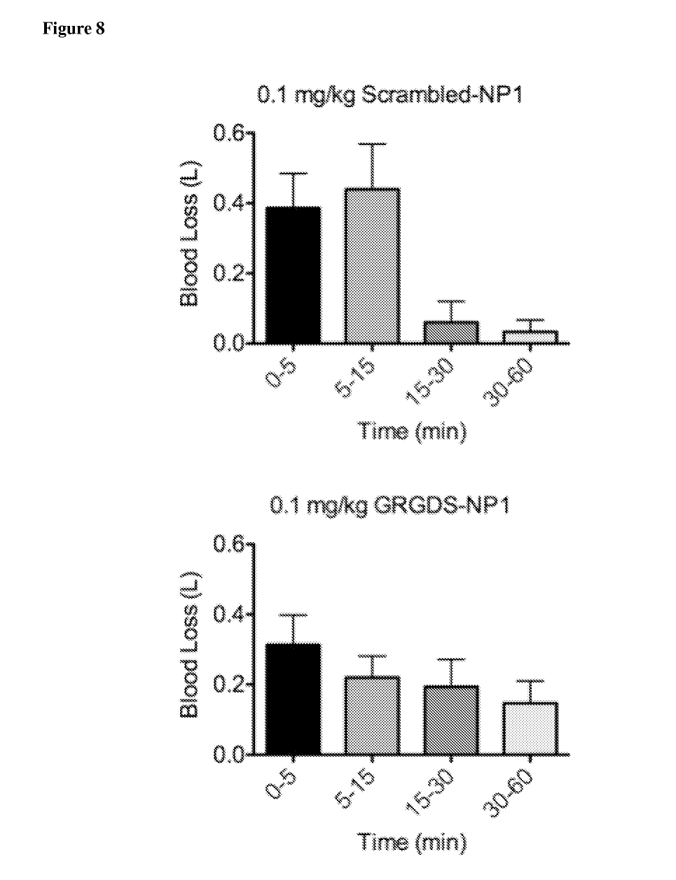

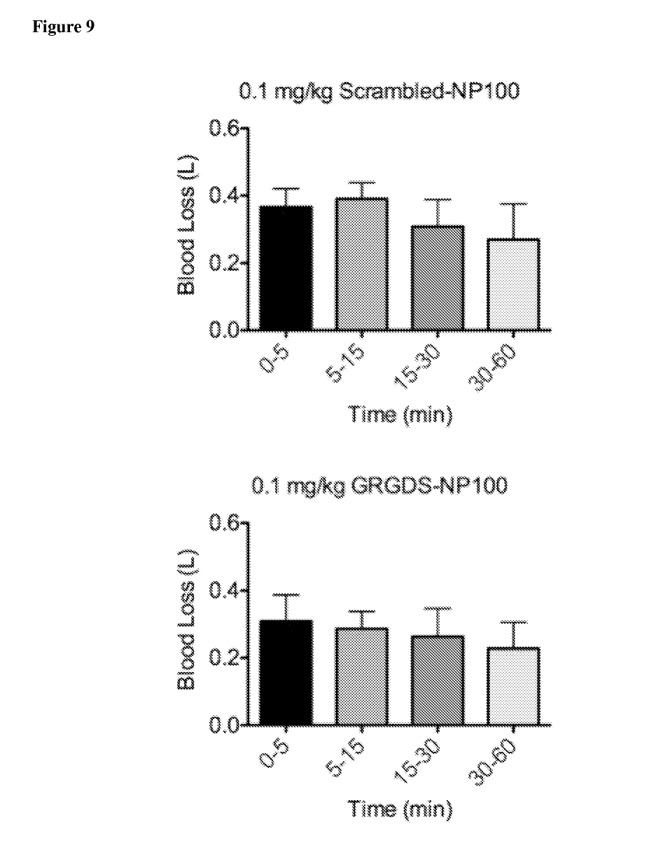

The initial hypothesis for this adverse response was that the particles may have been causing saturation of platelet receptors, as would be seen with administration of free RGD peptide, causing platelet inhibition. We therefore proceeded with our dosing study as planned, and found 0.1-0.2 mg/kg to be the “optimal” dose which did not elicit an adverse response. However, upon further analysis, the particles still appear to prolong bleeding times in the pigs, demonstrating increased amounts of bleeding post-treatment (5-60 min). This held true for both NP1 particles (FIG. 8) and NP100 particles (FIG. 9).

As shown in FIG. 8, while blood loss in the pre-administration (0-5 min) window was consistent between groups, the post-administration (5-60 min) blood loss was exacerbated greatly in the both the GRGDS (SEQ ID NO: 2) (560+018 ml) and scrambled (533+/−146 ml) groups compared to the saline control (395+004 ml). Mean survival time was 26 min for scrambled and 144 min for GRGDS (SEQ ID NO: 2), compared to 240 min for the saline control.

As shown in FIG. 9, while blood loss in the pre-administration (0-5 min) window was consistent between groups, the post-administration (5-60 min) blood loss was exacerbated greatly in the both the GRGDS (SEQ ID NO: 2) (777+077 ml) and scrambled (968+083 ml) groups compared to the saline control (395+004 ml). Mean survival time was 73 min for scrambled and 172 min for GRGDS (SEQ ID NO: 2), compared to 240 min for the saline control.

Several particle controls (2 mg/kg) that contained no targeting peptide were tested, suspecting that even the GRADSP (SEQ ID NO: 3) peptide may still be interacting with platelet receptors. However, it was observed that the nanoparticles induced a hemorrhagic response, regardless of the fact they contained no-peptide. Thus, the adverse effects are likely from a nonspecific interaction of the nanoparticles' material itself, leading to the development of a naive administration model to further investigate the phenomenon.

### Example 7

### Steroid Delivery of Synthetic Platelets in Full Body and Head Only Blast Trauma

Nanoparticles (PLGA-PLL-PEG-cRGD) were loaded with dexamethasome to investigate delivery of the drug using the nanoparticles as a delivery system using animal models of blast trauma.

### Physiology Response to Blast and Treatment—Weight Loss

Weight loss (g) of the rats was measured at 2 and 7 days after blast and compared to their weight on the day of testing. As expected, the sham groups (no blast) experience significantly less weight loss compared to the blast groups and there was no significant difference between the treatment groups. However, at seven days, the active group starts to show significant difference from the control and LR groups. This could demonstrate a physiological recovery after blast.

The sham was statistically significant compared to all other groups at 2 days. The sham group was significantly different than the control and LR groups. The active group (those receiving steroid-delivering synthetic platelets) than the controls was statistically significant compared to the control group (p<0.05) and is trending compared to the lactated ringers (LR) group (p=0.08).

### Neurobehavioral and Cognitive Assessments—Open Field

Animals that survived the seven day time point underwent cognitive and behavioral testing. In order to measure locomotor and exploratory behavior in rats the ‘Open Field Test’ was conducted (Sallinen et al., Br. J. Pharmacol. 150(4): 391-402 (2007)). Briefly, an opaque black acrylic box with dimensions 80×80×36 cm was used for the task. Animals were subjected to explore the box on 2^{nd} and 7^{th} day post blast exposure with no objects place with in the box. Activity changes were detected using EthoVision XT™ software tracking. Distance traveled and average velocity during the five minute task was obtained to detect the change in activity of animal after blast injury and treatment. The active group was statistically different from the LR group.

### NOR Assessment

In order to assess spatial learning and short term memory, the animals underwent a Novel Object Recognition (NOR) test. The well-established NOR test was used to gauge rodent memory (Bevins et al., Nat. Protoc. 1(3):1306-11 (2006), Davis et al., J. Neurosci. Methods 189(1): 84-7 (2010). Briefly, animals undergo an acclimation period two days prior to blast testing. This process was done to reduce stress and handling and increase familiarity with the testing environment (Besheer et al., Behav. Processes, 50(1): 19-29 (2000)). Seven days following blast exposure, the animals underwent two trials with a delay of 20 minutes between each trial for short term memory evaluation. The first trial (Ti) involved the exposure of animal to identical “familiar” objects for five minutes. In the second trial (T2), animals were exposed to a “familiar” object (same object used in the first task) and a “novel” object for five minutes. Trials and animal behavior were tracked using EthoVision XT™ tracking software. Precautions were taken to clean the chamber between the trials and have the experimenter leave the room during the experiment (Bevins et al., Nat. Protoc. 1(3): 1306-11 (2006)). For analysis, a discrimination index was calculated for each trial (time spent exploring the familiar object relative to the novel object divided by total time exploring objects during each trial). A ratio of 0.5 indicated equal exploration of both objects during the trial. Rats with entorhinal cortex lesions show poor discrimination of the novel objects (Aggleton et al., Behav. Neurosci. 124(1): 55-68 (2010)), thus this test can reflect damage to the entorhinal cortex and its role in memory formation as a portal to hippocampal processing. Results were provided with statistical analysis of each assessment.

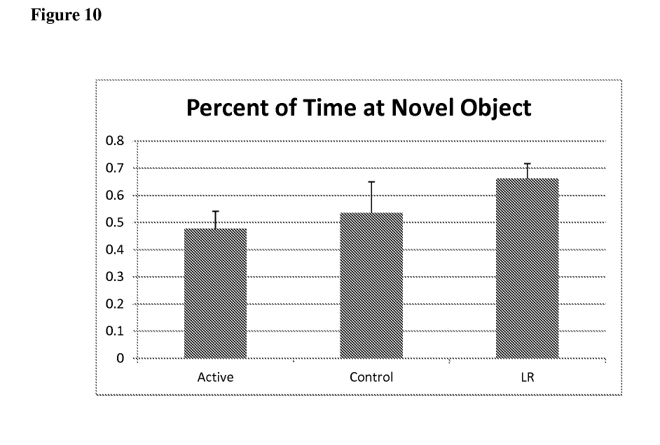

The results did not demonstrate a significant improvement of the short term memory deficits in the treatment group at one week following blast (FIG. 10). It is possible that the systemic recovery was delaying functional outcomes related to the cognitive centers of brain. As such, histological parameters were assessed.

Using the open field testing arena, animals were also assessed for anxiety-like behavior (Sallinen et al., Br. J. Pharmacol. 50(4): 391-402 (2007)). The open field consists of an empty arena. The innate tendency of a rat is to explore the open field, a tendency that is counterbalanced by a natural fear of open, lit spaces. Thus time spent along the chamber wall was thought to reflect an increased level of anxiety. Rats were videotaped for 5 minutes and avoidance of center square activity (i.e. anxiety-related behavior) was measured by determining the amount of time and frequency of entries into the central portion of the open field.

### Thigmotaxis Assessment—Anxiety in Rodents (3^{rd} Week Group—Steroid)

The active group was significantly different from both the control and LR groups (*-p<0.05) at seven days after blast. Prevalence for the walls was seen more in the control and LR groups. This work suggested that the steroid-loaded synthetic platelets may reduce anxiety and functional deficits associated with blast-induced head trauma.

### Histological Reponses to Blast and Treatment

After the one week survival time point and subsequent to behavioral tests, animals were euthanized and all critical organs were collected in fixative solution. Histological staining and analysis were completed on the lung and brain.

### Lung Injury

The lung tissue was analyzed for injury using 3 histological techniques. After 48 hours in fixative, the lungs were placed in 30% sucrose solution in order to prepare for tissue sectioning. Lungs were separated into cassettes with each lung lobe isolated for analysis. Samples from lobe A of the lung, determined as most injured following previous study, was cut and stained. Images were taken of three regions of interest (ROI) in each lung tissue section. These three images were converted to black and white and optical density readings were collected in order to determine the level of injury in the lung tissue using Image J software. The percent injured area was calculated in each lobe and significance was determined and reported as mean±SEM. Histological statistical analysis was calculated with a two way ANOVA followed by a post hoc LSD test with significance achieved with p<0.05.

First, lung tissue was assessed with the standard hematoxylin and eosin (H&E) stain. Below, the active group has trending significance versus the LR group. The results from other lobes are inconclusive as it is suspected that there is blood cell clearance by the one week time point.

### Brain Histopathology

The blast TBI studies have found histological markers of apoptosis and glial activity to be significantly elevated after blast exposure compared to controls (Sajja et al., NMR Biomedicine 25(12): 1331-9 (2012), Sajja et al., J. Neurosci. Res. 91(4): 593-601 (2013) and VandeVord et al. Ann. Biomed. Engin. 40(1): 227-36 (2012)). Thus, those markers were used to validate a mechanism for blast neurotrauma in our experimental lung injury model. Since reactive astrocytosis occurs prominently in response to all forms of central nervous system injury or disease¹, we examined the levels of GFAP within the brain tissue¹. Apoptotic cell death was confirmed by quantifying caspase 3 (Abcam, Cambridge, Mass.) which is an indicator of early stage apoptosis and FluoroJade B (Abcam, Cambridge, Mass.) which provides sensitive information about neuronal degeneration (Kim et al., BMC Neurosci, 10: 123 (2009)). Collectively, these stains will allow for the assessment of the presence, magnitude and nature of blast neural damage. All measures were scored individually to determine the correlation between staining, injury and recovery. Quantitative scores were compared across groups with ANOVAs.

### GFAP Expression in the Amygdala

GFAP expression, detected as green florescence, indicated the number of active astrocytes. A significant difference in the number of active astrocytes was observed in the active and control groups. The sham group was statistically different than all other groups. Integrated Density was normalized to area of image according to the amount of green fluorescence representing GFAP expression. Overall, it is clear that the sham and the active groups have fewer reactive astrocytes which are associated with brain trauma.

### Caspase-3 Expression in the Amygdala

Cleaved caspase-3 expression is a marker of cell death and it was measured in the amygdala. A significant difference in caspase-3 activity was observed in the control group compared to the active and sham groups. There was clearly more cell death in the control group and in the LR group than in the active and sham groups.

### FJB Expression

Florojade B is a marker for cell death in the brain. The marker was measured in the amygdale. The trend suggested that there was less death in the active and sham groups than the controls. The results were not significant due to the small sample size.

Overall, the histological analysis to date suggests that there is less cell death and fewer signs of trauma in the brain in the group that is receiving the steroid-delivering synthetic platelets (active group) than the controls, but the groups investigated were small.

### Example 7

The studies using steroid loaded nanoparticles demonstrate allowed for honing preferred concentrations of nanoparticles and poloxamer within the compositions of the invention. This is summarized in the Table 6 below. A composition comprising 20% poloxamer (weight by weight) to the nanoparticles. The addition of the poloxamer reduced aggregation and allowed for resuspension without sonication.

| mg poloxamer in a | Not Sonicated Diameter | | Sonicated Diameter | |
|---|---|---|---|---|
| 120 mg particle batch | Effective | Mean | Effective | Mean |
| 120 mg (50% wt/wt) | 404 | 366 | 307 | 104 |
| 30 mg (20%) | 938 | 774 | 380 | 366 |
| 10 mg (7.6%) | 988 | 823 | 471 | 394 |

What is claimed:

1. A spray composition comprising a co-block polymer coupled with a water soluble polymer, and a polymer delivery solvent.

2. The spray composition of claim 1 wherein the co-block polymer is a nanoparticle comprising a core, a water soluble polymer and a peptide.

3. The spray composition of claim 2 comprising a nanoparticle, wherein the nanoparticle comprises a water soluble polymer attached to the core at a first terminus of the water soluble polymer.

4. The spray composition of claim 2 or 3, wherein the peptide comprises an RGD amino acid sequence.

5. The spray composition of any one of claims 1-4 further comprising a polycation.

6. The spray composition of claim 5 wherein the polycation is positioned adjacent the co-block polymer and the water soluble polymer.

7. The spray composition of any one of claims 1-6, wherein the co-block polymer is a diblock copolymer, a triblock copolymer, an amphiphilic block copolymer or a PEG block co-polymer.

8. The spray composition of any one of claims 1-7, wherein the co-block polymer is poly(lactide-co-glycolide acid (PLGA), polylactic acid (PLA), polyglycolide (PGA), polycaprolactone (PCL), poly (ε-caprolactone), poly-L-lysine (PLL) or combinations thereof.

9. The spray composition any one of claims 1-8 wherein the water soluble polymer is selected from the group consisting of polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1, 3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof. In various aspects, the water soluble polymer is PEG having an average molecular weight between 100 Da and 10,000 Da or at least about 100.

10. The spray composition of any one of claims 1-9, wherein the water soluble polymer is PEG.

11. The spray composition of claim 10 wherein the PEG has an average molecular weight between 100 Da and 10,000 Da.

12. The spray composition of claim 10 or 11, wherein PEG has an average molecular weight of at least about 100.

13. The spray composition of any one of claims 1-12, wherein the water soluble polymer is attached to the core at a molar ratio of 0.1:1 to 1:10 or greater.

14. The spray composition of any one of claims 4-13 wherein the RGD peptide comprises a sequence selected from the group consisting of RGD, RGDS (SEQ ID NO: 1), GRGDS (SEQ ID NO: 2), GRGDSP (SEQ ID NO: 3), GRGDSPK (SEQ ID NO: 4), GRGDN (SEQ ID NO: 5), GRGDNP (SEQ ID NO: 6), GGGGRGDS (SEQ ID NO: 7), GRGDK (SEQ ID NO: 8), GRGDTP (SEQ ID NO: 9), cRGD, YRGDS (SEQ ID NO: 10) or variants thereof.

15. The spray composition of any one of claims 2-14, wherein the peptide is linear or cyclic.

16. The spray composition of claim 15, wherein the cyclic peptide is cyclic as a result of covalent association and/or the result of a conformation preference.

17. The spray composition of claim 4-16, wherein the RGD peptide is in a tandem repeat.

18. The spray composition of claim 4-16, wherein the RGD peptide is present in 2, 3, 4, 5, 6, 7, 8, 9, 10 or more copies of the RGD peptide

19. The spray composition of claim 18, wherein all of the RGD peptides are the same.

20. The spray composition of claim 18, wherein two copies of the RGD peptide have different sequences.

21. The spray composition of any one of claims 5-20, wherein the polycation is selected from polylysine, polyarginine, polyornithine, polyhistidine, cationic polysaccharides, POLYBRENE® (1,5-dimethyl-1,5-diazaundecamethylene polymethobromide, hexadimethrine bromide), histone, myelin basic protein, polymyxin B sulfate, dodecyltrimethylammonium bromide, bradykinin, spermine, putrescine, cadaverine, octylarginine, cationic dendrimer, and synthetic peptides.

22. The spray composition of any one of claims 5-21, wherein the polycation is polylysine.

23. The spray composition of any one of claims 1-22, wherein the polymer delivery solvent is dipolar aprotic solvent.

24. The spray composition of any one of claims 1-23, wherein the polymer delivery solvent is selected from the group consisting of dimethylsulfoxide (DMSO), N-Methyl-2-pyrrolidone (NMP), N,N dimethyl aceamide (DMF), and tetrahydrofuran (THF).

25. The spray composition of any one of claims 1-24, wherein the co-block polymer is PLGA.

26. The spray composition of any one of claims 1-25, wherein the peptide comprises the sequence GRGDS (SEQ ID NO: 2).

27. The spray composition of any one of claims 1-26, wherein the polymer delivery solvent is NMP.

28. The spray composition of any one of claims 1-27, wherein the water soluble polymer is PEG.

29. The spray composition of claim 5, wherein the co-block polymer is PLGA, the polycation is polylysine, the water soluble polymer is PEG, the peptide comprises the sequence GRGDS and the polymer delivery solvent is NMP.

30. The spray composition of any one of claims 1-29, wherein the water soluble polymer of having sufficient length to allow binding of the peptide to glycoprotein IIb/IIIa (GPIIb/IIIa), the composition further comprising a poloxamer.

31. The spray composition of claim 30, wherein the poloxamer is present at about 0.1% to about 60% of the composition.

32. The spray composition of claim 30 or 31, wherein the poloxamer in the composition is present up to 50 times nanoparticle mass.

33. The spray composition of any one of claims 30-32 wherein the poloxamer is a non ionic triblock copolymer comprising a structure -[hydrophilic polymer-hydrophobic polymer-hydrophilic polymer]n-.

34. The spray composition of any one of claims 30-33, wherein the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407 and Kolliphor P 188.

35. The compositions of any one of claims 1-34, wherein the nanoparticles have a spheroid shape and a diameter of less than 1 micron.

36. The spray composition of claim 35, wherein the nanoparticles have a diameter between 0.1 micron and 1 micron.

37. The spray composition of any one of claims 1-34, wherein the nanoparticles have a non-spheroid shape.

38. The spray composition of claim 37, wherein the nanoparticle is a rod, fiber or whisker.

39. The spray composition of claim 38, wherein the nanoparticle has an aspect ratio length to width of at least 3.

40. The spray composition of any one of claims 1-39, which is stable at room temperature for at least 14 days.

41. The spray compositions of any one of claims 1-40, wherein the nanoparticle core is crystalline polymer.

42. The spray composition of claim 41, wherein the core is a single polymer, a block copolymer, a triblock copolymer or a quadblock polymer.

43. The spray composition of any one of claims 1-42, wherein the nanoparticle core comprises PLGA, PLA, PGA, (poly (ε-caprolactone) PCL, PLL or combinations thereof.

44. The spray compositions of any one of claims 1-43, wherein the nanoparticle core is biodegradable.

45. The spray composition of any one of claims 1-44, wherein the nanoparticle core is solid.

46. The spray composition of any one of claims 1-43, wherein the nanoparticle core is non-biodegradable.

47. The spray composition of any one of claims 1-43, wherein the nanoparticle core is a material selected from the group consisting of gold, silver, platinum, aluminum, palladium, copper, cobalt, indium, nickel, ZnS, ZnO, Ti, TiO2, Sn, SnO2, Si, SiO2, Fe, Fe+4, steel, cobalt-chrome alloys, Cd, CdSe, CdS, and CdS, titanium alloy, AgI, AgBr, HgI2, PbS, PbSe, ZnTe, CdTe, In2S3, In2Se3, Cd3P2, Cd3As2, InAs, GaAs, cellulose or a dendrimer structure.

48. The spray composition of any one of claims 1-47, wherein the nanoparticle further comprises a therapeutic compound.

49. The composition of claim 48, wherein the therapeutic compound is hydrophobic.

50. The composition of claim 48, wherein the therapeutic compound is hydrophilic.

51. The spray composition of any one of claims 47-50, wherein the therapeutic compound is covalently attached to the nanoparticle, non-covalently associated with the nanoparticle, associated with the nanoparticle through electrostatic interaction, or associated with the nanoparticle through hydrophobic interaction.

52. The spray composition of any one of claims 47-51, wherein the therapeutic compound is a growth factor, a cytokine, a steroid, or a small molecule.

53. The spray composition of any one of claims 47-52, wherein the therapeutic compound is an anti-cancer compound.

54. A spray composition of any one of claims 1-53, which is a pharmaceutical composition.

55. A method of treating an condition in an individual comprising the step of administering a composition of any one of claims 1-54 to a patient in need thereof in an amount effective to treat the condition.

56. The method of claim 55, wherein the individual has a bleeding disorder.

57. The method of claim 56, wherein the composition is administered in an amount effective to reduce bleeding time by more than 15% compared to no administration or administration of saline.

58. The method of claim 56 or 57 wherein the bleeding disorder is a symptom of a clotting disorder, thrombocytopenia, a wound healing disorder, trauma, blast trauma, a spinal cord injury or hemorrhaging.
